(19) United States
(12) Patent Application Publication
(10) Pub. No.: US 2023/0374088 A1
(43) Pub. Date: Nov. 23, 2023

(54) PEPTIDE INHIBITORS OF GLIOMA-ASSOCIATED ONCOGENE DERIVED FROM THE COILED-COIL DIMERIZATION DOMAIN OF Kif7

(71) Applicant: The General Hospital Corporation, Boston, MA (US)

(72) Inventors: Radhika SUBRAMANIAN, Boston, MA (US); Farah HAQUE, Cambridge, MA (US); Christian Joseph FRENIERE, New Haven, CT (US); Qiong YE, West Roxbury, MA (US)

(21) Appl. No.: 18/030,876

(22) PCT Filed: Oct. 12, 2021
(86) PCT No.: PCT/US2021/054538

§ 371 Date: Apr. 7, 2023

Related U.S. Application Data
(60) Provisional application No. 63/089,770, filed on Oct. 9, 2020.

(51) Int. Cl. C07K 14/47

(52) U.S. Cl. CPC C07K 14/47; A61K 38/00

(57) ABSTRACT

The invention features a peptide comprising a coiled-coil dimerization domain which exploits DNA mimicry to engage Gli.

### CROSS-REFERENCE TO RELATED APPLICATIONS

This application claims benefit of U.S. Provisional Application No. 63/089,770, filed on Oct. 9, 2020, the contents of which are incorporated herein by reference in their entirety.

### SEQUENCE LISTING

The instant application contains a Sequence Listing which has been submitted electronically in ASCII format and is hereby incorporated by reference in its entirety. The ASCII copy, created on Oct. 1, 2021, is named 51545-002WO2_Sequence_Listing_10_1_21_ST25 and is 13,866 bytes in size.

### BACKGROUND OF THE INVENTION

An essential developmental pathway with a strict and conserved requirement for the microtubule cytoskeleton is Hedgehog (Hh) signaling (Robbins et al., Cell, 90, 225-234, 1997, Sisson et al., Cell, 90, 235-245, 1997, Wilson and Chuang, Development, 137, 2079-2094, 2010, Ingham et al., Nature Reviews Genetics, 12, 393-406, 2011). In this pathway, the principal effector and terminal transcription factor Gli (glioma-associated oncogene) is converted to either a full-length activator or a proteolytically cleaved repressor outside the nucleus (Jiang and Hui, Developmental cell, 15, 801-812, 2008, Hui and Angers, Annual review of cell and developmental biology, 27, 513-537, 2011). In vertebrates, Hh signaling pathway has a unique dependence on the primary cilium (Huangfu et al., Nature, 426, 83-87, 2003), a microtubule-based organelle. Gli is observed to traffic through the primary cilium upon pathway activation (He et al., Trends in cell biology, 27, 110-125, 2017), and defects in cilium assembly and architecture result in erroneous Gli processing (Caspary et al., Developmental cell, 12, 767-778, 2007, Goetz and Anderson, Nature Reviews Genetics, 11, 331-344, 2010). Therapeutic strategies are accordingly needed to target Gli in combatting cancer.

### SUMMARY OF THE INVENTION

We disclose a small coiled-coil dimerization domain in the ciliary kinesin Kif7 that binds to the DNA-binding domain of the Hedgehog signaling transcription factor Gli by mimicking the size, shape, and charge of DNA. The full-length sequence for the ciliary kinesin Kif7 is:

SEQ ID NO. 1:
MGLEA QRLPG AEEAP VRVAL RVRPL LPKEL
LHGHQ SCLQV EPGLG RVTLG RDRHF GFHVV
LAEDA GQEAV YQACV QPLLE AFFEG FNATV
FAYGQ TGSGK TYTMG EASVA SLLED EQGIV
PRAMA EAFKL IDEND LLDCL VHVSY LEVYK
EEFRD LLEVG TASRDI QLRED ERGN VVLCG
VKEVD VEGLD EVLSL LEMGN AARHT GATHL
NHLSS RSHTV FTVTL EQRGR APSRL PRPAP
GQLLV SKFHF VDLAG SERVL KTGST GERLK
ESIQI NSSLL ALGNV ISALG DPQRR GSHIP
YRDSK ITRIL KDSLG GNAKT VMIAC VSPSS
SDFDE TLNTL NYASR AQNIR NRATV NWRPE
AERPP EETAS GARGP PRHRS ETRII HRGRR
APGPA TASAA AAMRL GAECA RYRAC TDAAY
SLLRE LQAEP GLPGA AARKV RDWLC AVEGE
RSALS SASGP DSGIE SASVE DQAAQ GAGGR
KEDEG AQQLL TLQNQ VARLE EENRD FLAAL
EDAME QYKLQ SDRLR EQQEE MVELR LRLEL
VRPGW GGPRL LNGLP PGSFV PRPHT APLGG
AHAHV LGMVP PACLP GDEVG SEQRG EQVTN
GREAG AELLT EVNRL GSGSS AASEE EEEEE
EPPRR TLHLR RNRIS NCSQR AGARP GSLPE
RKGPE LCLEE LDAAI PGSRA VGGSK ARVQA
RQVPP ATASE WRLAQ AQQKI RELAI NIRMK
EELIG ELVRT GKAAQ ALNRQ HSQRI RELEQ
EAEQV RAELS EGQRQ LRELE GKELQ DAGER
SRLQE FRRRV AAAQS QVQVL KEKKQ ATERL
VSLSA QSEKR LQELE RNVQL MRQQQ GQLQR
RLREE TEQKR RLEAE MSKRQ HRVKE LELKH
EQQQK ILKIK TEEIA AFQRK RRSGS NGSVV
SLEQQ QKIEE QKKWL DQEME KVLQQ RRALE
ELGEE LHKRE AILAK KEALM QEKTG LESKR
LRSSQ ALNED IVRVS SRLEH LEKEL SEKSG
QLRQG SAQSQ QQIRG EIDSL RQEKD SLLKQ
RLEID GKLRQ GSLLS PEEER TLFQL DEAIE
ALDAA IEYKN EAITC RQRVL RASAS LLSQC
EMNLM AKLSY LSSSE TRALL CKYFD KVVTL
REEQH QQQIA FSELE MQLEE QQRLV YWLEV
ALERQ RLEMD RQLTL QQKEH EQNMQ LLLQQ
SRDHL GEGLA DSRRQ YEARI QALEK ELGRY
MWINQ ELKQK LGGVN AVGHS RGGEK RSLCS
EGRQA PGNED ELHLA PELLW LSPLT EGAPR
TREET RDLVH APLPL TWKRS SLCGE EQGSP
EELRQ REAAE PLVGR VLPVG EAGLP WNFGP
LSKPR RELRR ASPGM IDVRK NPL

The sequence of the small coil coiled dimerization domain that binds to Gli and is useful in the invention is:

SEQ ID NO. 2:
DSGIE SASVE DQAAQ GAGGR KEDEG AQQLL
TLQNQ VARLE EENRD FLAAL EDAME QYKLQ
SDRLR SEQRG EQVTN LRLEL VRPGW GGPRL
LNGLP PGSFV PRPHT APLGG AHAHV LGMVP
PACLP GDEVG EQQEE MVELR

We show how the peptide enumerated above and derived from the coiled-coil domain of Kif7 can be used as a tool to sequester Gli in the cytoplasm and inhibit its nuclear localization. We believe this is the first description of cytoplasmic DNA mimicry by a kinesin domain in eukayrotes. Our finding reveals this mechanism of regulating transcription factors by DNA-mimicry using a coiled-coil domain is widespread amongst cytoskeletal proteins where the coiled-coil domain is ubiquitous. These results disclose peptide inhibitors of the oncogene Gli (glioma-associated oncogene) that has been implicated in many humans cancers. This peptide derived from the Kif7 coiled-coil domain is useful as a therapeutic for inhibiting Gli-mediated transcriptional activity in cancers where Gli is aberrantly activated (for example, basal cell carcinoma and glioblastomas). Alternatively and advantageously, this peptide can be modified in the following different ways to increase its binding to endogenous Gli proteins, sequester Gli in the cytoplasm, and decrease the transcriptional activity of Gli:
A truncated coiled-coil domain that is sufficient for binding Gli and inhibiting its activity can be made. One such truncated version contains the following sequence:

SEQ ID NO. 3:
KEDEGAQQLLTLQNQVARLEEENRDFLAALED
AMEQYKLQSDRLREQQEEMVELRLRLELVRP

Amino acid mutations within these coiled-coil domains (SEQ. ID NOs: 2 and 3) can be made based on our results from point mutation analysis which elucidates the residues which lie at the contact surface of Kif7 and Gli and are essential for this interaction. Exemplary mutants include S1-mut (E500A, E501A, E502A, D505A), S2-mut (E511A, E515A), S3-mut1 (E526A, E529A) and S3-mut2 (E530A, R535A). (Numbering of the mutations is in the conventional manner using the full length sequence (SEQ ID NO:1)). Other exemplary truncations and sequences with mutations can been made and tested by one of skill in the art. See FIG. 3 and its legend for that analysis. This is within the level of skill in the art.

Peptides described herein range from about 60 to about 170 amino acids in length. Exemplary lengths include 50aa, 60aa, 70aa, 80aa, 90aa, 100aa, 110aa, 120aa, 130aa, 140aa, 150aa, 160aa, 170aa, and 180aa. Such peptides include a Gli binding region and a protein tag as discussed below.

Protein localization tags that would allow the specific localization of the coiled-coil peptide in different cell compartments and thus sequester Gli away from the nucleus into these compartments can be added as follows:
1. Endoplasmic reticulum (ER) tag—Amino acids KEDL are added at the C-terminus of Kif7 coiled-coil inhibitor. (see for example FIG. 7A, B, C)
2. Nuclear export signal (NES) tag—Amino acids IDMLIDLGLDLSD are added at the C-terminus of Kif7 coiled-coil inhibitor for example.
3. Mitochondrial localization (Mito) tag—Amino acids MLSLRQSIRFFKPATRTLCSSRYLL are added at the N-terminus of Kif7 coiled-coil inhibitor for example.
4. Add E3 ubiquitin ligase to its C-terminal end to ensure proteolytic degradation of Gli that is sequestered by the Kif7 coiled-coil peptide.

The Kif7 coiled coil inhibitor can be stabilized and optimized using stapled peptides to increase its pharmacological and therapeutic efficacy as an inhibitor. Accordingly, synthetic peptides based on the Kif7-coil coiled sequence can be generated by protein engineering that have high affinity for Gli-ZF binding (pM range). These peptides can be further stabilized by using a stapled peptide approach to improve their half-life in the body. For ease of delivery to the cells, membrane permeabilization tags and other protein conjugates (PEG, etc.) are employed. Additionally, synthetic Kif7 coiled-coil can be delivered in liposomes or as nanoparticle hybrids.

Additionally, a therapeutic transgene strategy may employed. Gene therapy based products (DNA/RNA/viral vectors) expressing a peptide disclosed herein (e.g., Kif7-coil coiled peptides) is useful as a cancer therapeutic. Such a strategy could also be used to direct specific expression of the product in cancerous tissue using specific promoter sequences

One of skill in the art can create any of the above modifications using standard methods known in the art. See, for example, Wuo, M. G., Hong, S. H., Singh, A. and Arora, P. S., 2018. Synthetic control of tertiary helical structures in short peptides. Journal of the American Chemical Society, 140(47), pp. 16284-16290 and Rezaei Araghi, R., Ryan, J. A., Letai, A. and Keating, A. E., 2016. Rapid optimization of Mci-1 inhibitors using stapled peptide libraries including non-natural side chains. ACS chemicalbiology, 11(5), pp. 1238-1244. Herein, the foregoing peptides (including SEQ ID NO: 2 and 3 and sequences modified as specified above) are termed “Kif7 coiled coil inhibitors” or “K7CCs”.

The Kif7 coiled coil inhibitors of the invention described herein can be used as a cell biological research tool to modify intracellular localization and downstream transcriptional activity of Gli, as well as in investigation of the Hedgehog signaling pathway. Iterative optimization of the Kif7 coiled coil inhibitors of the invention can produce a molecular tool to tether other zinc-finger domain containing transcription factors to the cytoplasmic cytoskeleton for regulating their activity. Further, the Kif7 coiled coil inhibitors of the invention can be employed as therapeutic agents in the treatment of cancers to inhibit Gli activity. Multiple cancers which result from over activation of Gli or the Hedgehog pathway are known, exemplary are basal cell carcinomas, medulloblastomas, glioblastomas, colorectal cancer, prostate cancer, lung cancer and breast cancer. These cancers can be treated with any one of the Kif7 coiled coil inhibitor of the invention. See, for example, Wu, F., et al., Hedgehog Signaling: From Basic Biology to Cancer Therapy. Cell Chem Biol, 2017. 24 (3): p. 252-280; Romer, J. and T. Curran, Targeting medulloblastoma: small-molecule inhibitors of the Sonic Hedgehog pathway as potential cancer therapeutics. Cancer Res, 2005. 65 (12): p. 4975-8; Pak, E. and R. A. Segal, Hedgehog Signal Transduction: Key Players, Oncogenic Drivers, and Cancer Therapy. Dev Cell, 2016. 38 (4): p. 333-44; and Didiasova, M., L. Schaefer, and M. Wygrecka, Targeting GLI Transcription Factors in Cancer. Molecules, 2018. 23 (5).

By “substantially identical” is meant a peptide or nucleic acid exhibiting at least 40%, 50%, 60%, 70% 80%, 90%, or even 95%, 96%, 97%, 98% or 99% sequence identity to a reference sequence (for example, the amino acid sequences of SEQ ID NO:2 or 3 or to their respective nucleic acid sequences).

For polypeptides, the length of comparison sequences will generally be at least 16 amino acids, preferably at least 20 amino acids, more preferably at least 25 amino acids, and most preferably 35 amino acids or greater. For nucleic acids, the length of comparison sequences will generally be at least 50 nucleotides, preferably at least 60 nucleotides, more preferably at least 75 nucleotides, and most preferably 110 nucleotides or greater.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, FastA, or PILEUP/PRETTYBOX programs). Such software matches identical or similar sequences by assigning degrees of homology to various substitutions, deletions, and/or other modifications. Conservative substitutions typically include substitutions within the following groups: glycine alanine; valine, isoleucine, leucine; aspartic acid, glutamic acid, asparagine, glutamine; serine, threonine; lysine, arginine; and phenylalanine, tyrosine. Sequence identity is typically ascertained over the entire sequence of reference sequence such as SEQ ID NO:2 or SEQ ID NO:3.

Those skilled in the field of molecular biology will understand that any of a wide variety of vector expression systems may be used to provide the peptides described herein. Regulatory transcript regions may also be provided in such vector systems (e.g., DNA or RNA) according to standard methods known in the art.

Other features and advantages of the invention will be apparent from the following description of the preferred embodiments thereof, and from the claims.

### BRIEF DESCRIPTION OF THE DRAWINGS

FIG. 1A shows the elucidation of the Kif7 domains that interact with Gli2. Pull-down of c-Myc-Gli2 (1-594aa) and FLAG-Kif7 constructs (i to vi) after co-transfection in Expi293F cells (left panel). Domain architecture of full length Kif7 and deletion constructs used in this experiment (right panel). Immunoprecipitation (IP) using anti-FLAG magnetic beads. Input (cell lysate), FT (flow through), wash and beads samples were immunoblotted (IB) with anti c-myc antibody to detect Gli2. Transfection with c-Myc-Gli2 (1-594aa) alone was included as a negative control. The blots are representative images of a minimum of three repeats (center panel). Summary of results. The +/− sign indicates binding/no binding.

FIG. 1B shows the elucidation of the Gli2 domains that interact with Kif7. Pull-down of c-Myc-Gli2 constructs (i to vi) with different FLAG-Kif7 constructs after co-transfection in Expi293F cells. Kif7(1-543aa) was used for i) & ii), Kif7(362-600aa) was used for iii) & iv) and Kif7(460-600aa) was used for v) & vi) (left panel). Domain architecture of full-length Gli2 and deletion constructs used in this experiment (right panel). Immunoprecipitation (IP) using anti-FLAG magnetic beads. Input (cell lysate), FT (flow through), wash and beads samples were immunoblotted (IB) with anti c-myc antibody to detect Gli2. Transfection with each of the different c-Myc-Gli2 constructs alone were included as negative controls. The blots are representative images of a minimum of three repeats (center panel). Summary of results. The +/− sign represents presence/absence of binding.

FIG. 1C shows a Bio-Layer Interferometry (BLI) assay to quantitatively examine the binding affinity of Kif7 coiled-coil domain constructs; GST-Kif7-CC (460-600aa; black) & GST-Kif7-SCC (481-543aa; blue), to Gli2-ZF. Data represent mean and standard deviation from three independent repeats. The plots of binding response versus Gli2-ZF concentration were fit to a Hill equation to determine equilibrium dissociation constants (K_{d}). For Kif7-CC (460-600aa): K_{d}=48±5 nM, for Kif7-SCC (481-543aa): K_{d}=69±20 nM. See also FIGS. 8A & 8B.

FIG. 1D shows chromatograms from size exclusion chromatography of a mixture of 125 μM Kif7-CC-GFP and 750 μM Gli2-ZF-GFP (Superdex 200 10/300 GL). SDS-PAGE of the complex (inset) was used to determine stoichiometry by in-gel GFP fluorescence analysis and is indicated in parenthesis. Gel image is representative of a minimum of three repeats. See also FIGS. 8C, 8D & 8E.

FIG. 2A shows a structural model of Gli2-ZF (ribbon diagram; SWISS MODEL server: GMQE=0.98, QMean=−5.14) and overall electrostatic surface representation of the model.

FIG. 2B shows a structural model of Kif7-CC (ribbon diagram; SWISS MODEL server: GMQE=0.55, QMean=1.84) and overall electrostatic surface representation of the model.

FIG. 2C shows a Docked model of the Kif7-CC-Gli2-ZF protein complex (ClusPro 2.0) and overall electrostatic surface representation of the complex (left panel). A structural model of Gli2-ZF bound to DNA (based on Gli1-ZF-DNA crystal structure; PDB: 2GLI) is included for comparison of the shape and size of the two complexes (right panel). Dotted lines represent molecular dimensions of Kif7-CC and DNA. See also FIG. 9.

FIG. 3A shows binding of Gli2-ZF and Kif7-CC in the absence (black) and presence of 125 nM sequence-specific Gli2 target dsDNA (purple). Normalized binding response from the BLI measurements is plotted against Gli2-ZF concentration. Data represent mean and standard deviation from three independent repeats.

FIG. 3B shows the effect of increasing ionic strength (KCl concentration) on the Kif7-CC and Gli2-ZF binding. Normalized binding response was measures at 100 nM Gli2-ZF using the BLI assay. Data represent mean and standard deviation from three independent repeats. Yellow bar marks physiological ionic strength (˜125-150 mM KCl).

FIG. 3C shows a schematic of Gli2-ZF truncations and summary of their binding to Kif7-CC (left panel). Raw traces from the association step in the BLI assay performed with soluble 125 nM Gli2-ZF truncations and Kif7-CC (right panel). Equilibrium dissociation constants K_{d} (shown in parenthesis) were determined by fitting complete binding curves to Hill equation (See FIG. 10C). See also FIGS. 10A, 10B & 10C. Data is representative trace of a minimum of three repeats.

FIG. 3D shows a pull down experiment with c-Myc-Gli2 (418-594aa) and FLAG-Kif7-CC (460-600aa) mutant proteins after co-transfection in Expi293F cells. Immunoprecipitation (IP) using anti-FLAG magnetic beads. Input (cell lysate), FT (flow through), wash and beads samples were immunoblotted (IB) with anti c-Myc antibody to detect Gli2. Cells that were only transfected with c-Myc-Gli2 (418-594aa) were included as a negative control. The blots are representative images of a minimum of three repeats. Gli2-ZF mutants are as follows: ZF2-mut (H493A, R496A), ZF3-mut (H503A, R516A, K521A), ZF4-mut (R550A, K552A, R556A). Kif7-CC mutants are as follows: S1-mut (E500A, E501A, E502A, D505A), S2-mut (E511A, E515A), S3-mut1 (E526A, E529A) and S3-mut2 (E530A, R535A).

FIG. 3E shows a structural model of Kif7-SCC:Gli2-ZF protein complex. In red are residues on Gli2-ZF which when mutated to alanine abolish binding with Kif7-CC. In dark blue are residues on Kif7-CC which when mutated to alanine abolish binding to Gli2-ZF. In yellow are residues on both Kif7-CC and Gli2-ZF which when mutated to alanine did not abolish binding. Sections highlighted in green (S1, S2, S3) represent 3 patches of residues on Kif7-CC that were predicted as potential Gli binding sites by PDBsum analysis.

FIG. 4A shows a schematic of the in vitro total internal reflection fluorescence (TIRF) microscopy-based assay used to examine microtubule-localized Gli2-ZF and Kif7-DM. Rhodamine/HiLyte 647 labeled GMPCPP-stabilized microtubules (grey) were immobilized on a PEG-treated glass coverslip (yellow) via neutravidin-biotin linkages (blue & brown respectively). Kif7-DM-GFP (green), Gli2-ZF-SNAP-Alexa 647 (pink) and 1 mM ATP were subsequently added to examine binding of both proteins on microtubules. All proteins for TIRF microscopy assay were purified to >95% purity (FIGS. 11A & 11B). See also FIGS. 11C-11D.

FIG. 4B shows representative images of microtubule (MT, top) and Gli2-ZF-Alexa 647 (Gli2, bottom, 100 nM) in the absence (left) or presence of Kif7-DM-GFP (right, 100 nM). The graphs below show line scans of the corresponding Alexa 647 fluorescence intensity. Scale bars represent 2 μm.

FIG. 4C shows representative images of microtubule (MT, top) and Kif7-DM-GFP (Kif7, bottom, 100 nM) in the absence (left) or presence of Gli2-ZF (right, 50 nM). The graphs below show line scans of the corresponding GFP fluorescence. Scale bars represent 2 μm.

FIG. 4D shows a scatter plot of Kif7-DM-GFP intensity per pixel on microtubules (MT) in the presence of increasing concentrations of Gli2-ZF. Assay conditions: 100 nM Kif7-DM-GFP with 0, 50, 100, 200, 300 & 600 nM Gli2-ZF. N>90 at every Gli2-ZF concentration. Data fit to Hill equation (dotted grey line) shows saturation of the dose response at 600 nM Gli2-ZF and half maximal response at −160 nM Gli2-ZF.

FIG. 4E shows representative kymographs to visualize single molecules of Kif7-DM-GFP (1 nM) on X-rhodamine labeled microtubules with increasing Gli2-ZF concentrations (0, 10, 25 & 50 nM).

FIG. 5A shows a scatter plot of Gli2-ZF-Alexa647 intensity per pixel on microtubules (MT) represents recruitment of Gli on MT by Kif7-MM and Kif7-DM. The no kinesin condition was included as a control for non-specific binding of Gli2-ZF to MT and Kif27-DM was included as a negative control for Gli2-ZF binding. Assay conditions: 100 nM Gli2-ZF-SNAP-Alexa647 with 100 nM of kinesin in each case. N>50 for each condition.

FIG. 5B shows chromatograms from size exclusion chromatography of a mixture of 125 μM Kif7-DM-GFP and 750 μM Gli2-ZF-GFP (Superdex 200 10/300 GL). SDS-PAGE of the complex (inset) was used to determine stoichiometry by in-gel GFP fluorescence analysis and is indicated in parenthesis. Gel image is representative of a minimum of three repeats. See also Figure S4A-D.

FIG. 5C shows a Cryo-EM reconstruction of Kif7-DM bound to microtubules in the presence of Gli2-ZF-SNAP and AMPPNP. The structural model for Kif7 motor domain (green ribbon) bound to microtubule (grey ribbon) was fitted in the density and refined. The density corresponding to Gli2-SNAP is shown in pink. AMPPNP is shown as a yellow ball-and-stick model. See also FIGS. 12A-12E.

FIG. 5D shows Comparison of electrostatic surface potential of coiled-coil and motor domains of Kif7 (PDB: 6 MLR) and Kif27. The structural models of Kif7-CC (SWISS MODEL server: GMQE=0.55, QMean=1.84), Kif27-CC (SWISS MODEL server: GMQE=0.53, QMean=0.80) and Kif27 motor domain (SWISS MODEL server: GMQE=0.47, QMean=−2.22) were obtained by homology modelling. Loop L6, helices α-2 and α-3 on the Kif7 motor domain that lie close to the Gli2-SNAP density are highlighted in green are present a predominantly negative surface charge. Corresponding regions (Loop L6, helices α-2 and α-3) in the Kif27 motor domain model are highlighted in brown and show a neutral surface potential.

FIG. 5E shows schematics showing the domains swapped in the Kif7-Kif27 chimera proteins. Scatter plot of Kinesin-GFP intensity per pixel on microtubules (MT) in the presence of increasing concentrations of Gli2-ZF. Assay conditions: 100 nM of each kinesin: Kif7-DM (black), Kif7M-Kif27CC (red) and Kif27M-Kif7CC (blue), with 0, 100, 300 & 600 nM Gli2-ZF. N>40 at every Gli2-ZF concentration. Data were fit to Hill equation (dotted lines).

FIG. 6A shows localization of transfected mRuby-tagged full length Kif7 and mNeonGreen-tagged full length Gli2 in HeLa cells. Representative images are shown. DAPI staining was used to mark the nucleus. Overexpressed full length Gli2 shows predominant localization in the nucleus (row1). Overexpressed full length Kif7 is cytoplasmic (row2). Co-transfection with Kif7 and Gli2 shows localization of both proteins on microtubules in the cytoplasm (row3). Scale bars represent 10 μm.

FIG. 6B shows cilium-tip localization of Kif7 in WT, Gli2−/−, GIi2−/−Gli3−/− and Kif7−/− MEFs. Representative immunofluorescent images of primary cilia are shown. Acetylated α-tubulin antibody was used to mark cilia, γ-tubulin antibody was used to mark centrioles (base of cilia) and Alexa-Fluor-647-labeled Kif7 antibody was used to measure Kif7 amounts in the cilia tips during Hh activation (+SAG). Scale bar represents 2 μm.

FIG. 6C shows a quantitative analysis of the levels of Kif7 in WT, Gli2−/−, Gli2−/−Gli3−/− and Kif7−/− MEFs. Line scans of cilia from the experiment in FIG. 6B was used to quantify distribution of Kif7 along the length of the cilia. Data represent mean and standard error from three independent repeats (N>10 for each genotype).

FIG. 7A shows the localization of transfected mRuby-tagged Kif7-CC (460-600aa) with ER localization signal and mNeonGreen-tagged full length Gli2 in HeLa cells. Representative images are shown. DAPI staining was used to mark the nucleus. Overexpressed full length Gli2 shows predominant localization in the nucleus (row1). Co-transfection with Kif7-CC and Gli2 shows co-localization of Gli2 in the cytoplasm with Kif7-CC (row2). Scale bars represent 10 μm.

FIG. 7B shows the percent of nucleus-localized Gli2 as quantified from FIG. 7A in the absence and presence of Kif7-CC. Data represent mean and standard deviation from three independent repeats (N>35 for each condition).

FIG. 7C shows a Scatter plot of percent nuclear Gli2 versus level of Kif7-CC expression. The dotted line represents fit of the data to one-phase association.

FIG. 7D shows a schematic of the structural basis and cellular implications of Kif7-Gli interactions. 7D(i) shows DNA molecular mimicry by the Kif7 coiled-coil underlies the tethering of Gli in the cytoplasm and the cilia tips. The zinc-finger domain of Gli that binds DNA in the nucleus is co-opted for binding the Kif7 coiled-coil out of the nucleus. 7D(ii) shows the Kif7 coiled-coil domain acts as a molecular rheostat for the graded accumulation of both the proteins on microtubules and at the cilium tip in a Gli-concentration responsive manner. Consistent with this, Gli knockout cells (Gli−/−) have low levels of Kif7 at the cilia tips compared to WT cells during Hedgehog signal transduction. In Kif7 knockout cells (Kif7−/−) Gli is distributed in puncta along the length of the ciliary axoneme and is not concentrated at the cilia tip during pathway activation (He et al., 2014, Liu et al., 2014). 7D(iii) shows the Kif7 coiled-coil peptide can be re-tooled as a Gli sequestration agent in the cytoplasm to decrease nuclear Gli levels.

FIG. 8A shows chromatograms from size exclusion chromatography of GST-Kif7-CC-GFP (dark blue), GST-Kif7-CC (green), GST-Kif7-SCC (light blue), Gli2-ZF-GFP (red) and Gli2-ZF (maroon) on Superdex 200 10/300 GL. Arrows indicate the elution volumes of the following standards: (left to right) 1-ferritin (440 kDa), 2-aldolase (158 kDa), 3-ovalbumin (44 kDa) and 4-carbonic anhydrase (29 kDa).

FIG. 8B shows a SDS-PAGE of purified GST-Kif7-CC-GFP, GST-Kif7-CC, GST-Kif7-SCC, Gli2-ZF-GFP and Gli2-ZF. MW—molecular weight markers.

FIG. 8C shows a single molecule fluorescence intensity histograms of Gli2-ZF-GFP (Intensity=3.0×10⁴±1.4×10⁴, N=746) and GST-tagged Kif7-CC-GFP (Intensity=4.2×10⁴±1.9×10⁴, N=536). Intensities are reported as mean±standard deviation.

FIG. 8D shows a single molecule photobleaching traces for Gli2-ZF-GFP (1^{st} row) and GST-Kif7-CC-GFP (2^{nd} row). Background subtracted integrated fluorescence intensity versus time plots used for step photobleaching analysis. Photobleaching steps are indicated by arrows.

FIG. 8E shows a BLI assay to quantitatively examine the binding affinity of Kif7-CC-GFP to Gli2-ZF (black) & Gli2-ZF-GFP (green). Data represent mean and standard deviation from three independent repeats. The plots of binding response versus Gli2-ZF concentration were fit to a Hill equation to determine equilibrium dissociation constants (K_{d}). For Kif7-CC-GFP+Gli2-ZF: K_{d}=65±22 nM, for Kif7-CC-GFP+Gli2-ZF-GFP: K_{d}=57±9 nM. GFP-tagged versions of proteins show a consistent increase in K_{d} that is within error range.

FIG. 8F shows a Western blot of peak complex fraction from size exclusion chromatography of Kif7-CC-GFP and Gli2-ZF-GFP (from FIG. 1D). Stoichiometry of components in complex is indicated in parenthesis.

FIG. 9A shows another structural model.

FIGS. 9B-9E show structural models that are eliminated by mutagenesis experiments in FIGS. 3C-E (specifically Kif7-CC S2-mut, S3-mut1 and S3-mut2).

FIGS. 9F-9G shows structural models that are eliminated by the DNA competition assay in FIG. 3A.

FIG. 10A shows chromatograms from size exclusion chromatography of the Gli2-ZF truncation proteins on Superdex 75 10/300 GL.

FIG. 10B shows a SDS-PAGE of purified Gli2-ZF truncation proteins. MW—molecular weight markers.

FIG. 10C shows a binding response in BLI assay for the binding of Kif7-CC with Gli2 truncation constructs: ZF1-3 and ZF1-4. Data represent mean and standard deviation from three independent repeats. The plots of binding response versus Gli2-ZF concentration were fit to a Hill equation to determine equilibrium dissociation constants (K_{d}). For Gli2-ZF 1-4: K_{d}=103±15 nM and Gli2-ZF 1-4: K_{d}=160±40 nM.

FIG. 11A shows chromatograms from size exclusion chromatography of Kif7-DM (black), Kif7-DM-GFP (green) and Gli2-ZF-SNAP (magenta, * shows the peak that was used for experiments) on Superdex 200 10/300 GL. Dotted line indicates elution volume of Gli2-ZF on the same column. Arrows indicate the elution volumes of the following standards: (left to right) 1-ferritin (440 kDa), 2-aldolase (158 kDa) and 3-ovalbumin (44 kDa).

FIG. 11B shows a SDS-PAGE of purified Kif7-DM, Kif7-DM-GFP and Gli2-ZF-SNAP. MW13 molecular weight markers.

FIG. 11C shows fluorescence intensity histograms of Kif7-MM-GFP (Intensity=3.0×10⁴±1.7×10⁴, N=648) and Kif7-DM-GFP (Intensity=4.0×10⁴±2.0×10⁴, N=1015). Intensities are reported as mean±standard deviation.

FIG. 11D shows a single molecule photobleaching traces for Kif7-MM-GFP (top) and Kif7-DM-GFP (bottom). Background subtracted integrated fluorescence intensity versus time plots used for step photobleaching analysis. Photobleaching steps are indicated by arrows.

FIG. 12A shows a Fourier shell correlation (FSC) curve for Kif7-DM bound to GMPCPP microtubules in the presence of AMPPNP and Gli2-ZF-SNAP.

FIG. 12B shows a Cryo-EM reconstruction of dimeric Kif7 motor domain (green) in complex with AMPPNP bound on the GMPCPP-microtubule lattice (grey) in the presence of Gli2-ZF-SNAP (pink), shown in two orientations and from the −end and +end of the microtubule. Density of Gli2-ZF-SNAP is seen only attached to the Kif7 motor domain and no extra density is seen along the microtubule lattice.

FIG. 12C shows EM reconstructions of AMPPNP-bound Kif7-DM with Gli2-ZF-SNAP (black mesh) and AMPPNP-bound Kif7-MM with Gli2-ZF-SNAP (orange) superposed via the Kif7 motor domain. Structural model for AMPPNP-bound Kif7 motor (green) is shown.

FIG. 12D shows EM reconstructions of AMPPNP-bound Kif7-DM with Gli2-ZF-SNAP (black mesh) and ADP-bound Kif7-DM with Gli2-ZF (yellow) superposed via the Kif7 motor domain. Structural model for AMPPNP-bound Kif7 motor (green) is shown. Blue dotted region indicates density seen only with Gli2-ZF-SNAP and not with Gli2-ZF (thereby corresponding to part of SNAP peptide).

FIG. 12E shows Cryo-EM reconstructions of AMPPNP-bound Kif7-DM with Gli2-ZF-SNAP. Structural models for AMPPNP-bound Kif7 motor (green) and Gli2-ZF consisting of 2 complete and one partial zinc finger (magenta) are shown. Helix α-2 (dark green) and loop L2 (blue) of Kif7 make contacts with the density for Gli2-ZF-SNAP.

FIG. 12F shows chromatograms from size exclusion chromatography of Kif7M-Kif27CC (red) and Kif27M-Kif7CC (blue) on Superdex 200 10/300 GL. Dotted line indicates elution volume of Kif7-DM on the same column.

FIG. 12G shows a SDS-PAGE of purified Kif7M-Kif27CC and Kif27M-Kif7CC. MW—molecular weight markers.

FIG. 12H shows binding of GFP-tagged Kif7-DM (black), Kif7M-Kif27CC (red) and Kif27M-Kif7CC (blue) to Gli2-ZF measured using BLI. Data represent mean and standard deviation from three independent repeats. The plots of binding response versus Gli2-ZF concentration were fit to a Hill equation to determine equilibrium dissociation constants (K_{d}). For Kif7-DM+Gli2-ZF: K_{d}=217±70 nM; Kif7M-Kif27CC+Gli2-ZF: K_{d}=397±88 nM and Kif27M-Kif7CC+Gli2-ZF: 213±65 nM.

FIG. 12I shows recruitment of Gli to microtubules (MT) by Kif7-DM (black) and the two chimeras: Kif7M-Kif27CC (red) and Kif27M-Kif7CC (blue). Scatter plot shows the ratio of Gli2-ZF-Alexa647 intensity to Kinesin GFP intensity on microtubules. Assay conditions: 100 nM Gli2-ZF-SNAP-Alexa647 with 100 nM of kinesin in each case. N>40 for each condition.

FIG. 13A shows representative immunofluorescent images of cilium-tip localization of endogenous Gli2 in NIH3T3 cell transfected with mRuby-tagged Kif7-CC (460-600aa) with ER retention tag (top row) and untransfected cell from the same sample (bottom row). Acetylated α-tubulin antibody was used to mark cilia, γ-tubulin antibody was used to mark centrioles (base of cilia) and Alexa-Fluor-647-labeled Gli2 antibody was used to measure endogenous Gli2 amounts in the cilia tips during Hh activation (+SAG). Scale bar in the left panel represents 10 μm and in the cilium inset represents 2 μm.

FIG. 13B shows a quantitative analysis of the levels of Gli2 in cilia of NIH3T3 cells transfected with Kif7-CC-ER in the presence of SAG. Line scans of cilia from FIG. 13B was used to quantify distribution of Gli2 along the length of the cilia. Data represent mean and standard error from three independent repeats (N>12 cilia for each group). Two-way ANOVA (factor 1: transfected with Kif7-CC-ER p<0.05, factor 2: distance from tip p<0.0001) and post-hoc analysis (*p<0.0001 in unpaired Student's t-test) shows statistically significant differences in Gli2 intensity in cilia tips upon Kif7-CC-ER transfection compared to non-transfected control.

### DETAILED DESCRIPTION

The mechanisms underlying localization of the Hedgehog pathway effector Gli to microtubules for its cytoplasmic localization and regulation have been examined. We focused on the interaction of Gli with the conserved pathway protein, the ciliary kinesin Kif7. Surprisingly, we found that, unlike other kinesins that employ the canonical C-terminus cargo-binding domain to engage binding partners, Kif7 uses a small coiled-coil dimerization domain at the N-terminus to form a high-affinity interaction site for the DNA binding zinc-finger domain of Gli2. Homology modeling revealed a striking size, shape and electrostatic similarity between Kif7 coiled-coil dimerization domain and the DNA, suggesting that Kif7 exploits DNA mimicry to engage Gli2. This is not only the first description of DNA mimicry in the eukaryotic cytoplasm, but also one by a kinesin. Our Total Internal Reflection Fluorescence (TIRF) microscopy analysis of the Kif7-Gli interaction on microtubules showed that Kif7 and Gli synergistically accumulated on microtubules in a graded fashion depending on Gli-concentration. We examined Kif7-Gli interactions using cryo-EM and observed that this occurs through concomitant binding of a second Gli molecule to the Kif7 motor domain. Collectively, our results indicate that, in contrast to proposed models, Kif7 is not a passive platform for Gli binding but an active regulator of Gli that employs a highly unusual structural mechanism, DNA-mimicry, for transcription factor tethering in the cytoplasm. We show that the DNA-mimicry can be co-opted to engineer tools for sequestering Gli in the cytoplasm, thus revealing the potential to develop it as a therapeutic tool for inhibition of Gli when it is aberrantly activated in certain human cancers.

The following examples are put forth to provide those of ordinary skill in the art with a description of how the compositions and methods claimed herein are performed, made, and evaluated, and are intended to be purely exemplary described herein and are not intended to limit the scope of any invention disclosed herein.

The DNA-Binding Domain of GIi2 Forms a High Affinity Complex with the Dimerization Domain of Kif7

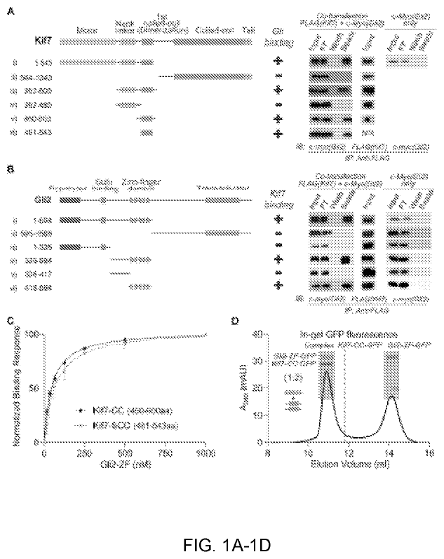

Gli proteins have been shown to co-immunoprecipitate with Kif7 in pull-down assays from mouse embryo lysates (Cheung et al., 2009. Science signaling, 2, ra29-ra292009), however biochemical and structural characterization of these complexes has been hampered by the proteolytic sensitivity and low solubility of these large (>150 kDa) multi-domain proteins (FIGS. 1A and 1B). Kif7 is a 1343 amino acid long kinesin consisting of the ATPase (motor) domain at the N-terminus, followed by an unusually long neck linker, a short coiled-coil dimerization domain and the canonical cargo binding domain at the C-terminus (FIG. 1A). Gli2 is a 1568 amino acid long transcription factor that consists of a highly conserved central DNA binding domain featuring 5 zinc-finger repeats, flanked by the N-terminal repressor and C-terminal activator domains (FIG. 1B). To determine the minimal interacting domains of Kif7 and Gli2 we performed pull-down assays with transiently over-expressed FLAG-tagged Kif7 and c-Myc-tagged Gli2 constructs in human Expi293F cells. Consistent with the results from mouse embryo lysates, we observed that the N-terminal half of Kif7 (1-543aa; which forms a dimer and hereafter referred to as Kif7-DM) interacts with the N-terminal half of Gli2 (1-594aa) (FIGS. 1Ai & Bi). No interaction was detected between the C-terminal half of Kif7 (544-1343aa) and the N-terminal half of Gli2 (1-594aa) (FIG. 1Aii) or the C-terminal half of Gli2 (595-1568aa) and the N-terminal half of Kif7 (1-543aa) (FIG. 1Bii).

We used the pull-down assay to map the minimal domains in both Gli2 and Kif7 that are sufficient for interaction. The interaction site on Kif7 was mapped to its first coiled-coil dimerization domain flanked by putative unstructured linkers (460-600aa; hereafter referred to as Kif7-CC) (FIG. 1Av). The region of interaction sufficient for binding could be further narrowed to a 63 amino acid shorter coiled-coil segment of Kif7-CC (481-543aa; hereafter referred to as Kif7-SCC) (FIG. 1Avi). Unexpectedly, we find that Gli binds Kif7 via its DNA-binding domain (418-594aa) (FIG. 1Bvi).

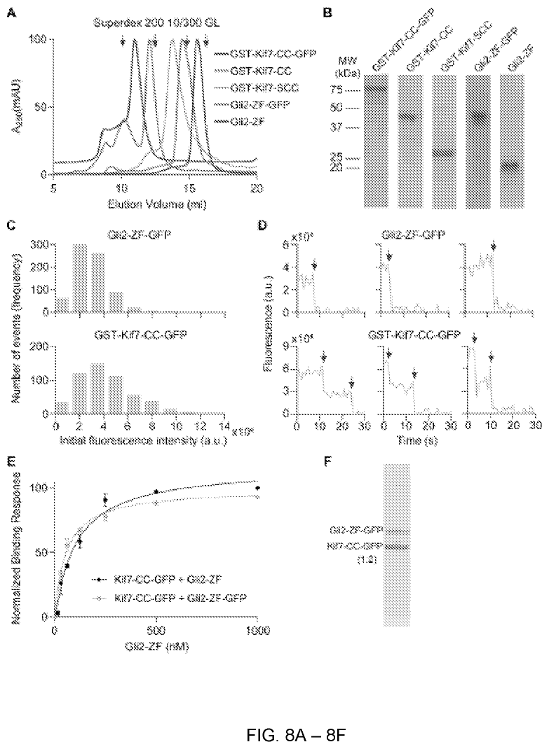

To confirm that Kif7 and Gli directly bind each other and determine the dissociation constant of the complex, we performed binding assays with purified recombinant Gli2-ZF (418-604aa; zinc finger domain of Gli2) and two Kif7 constructs: Kif7-CC (460-600aa), Kif7-SCC (481-543aa) (FIGS. 8A & 8B). Single molecule fluorescence intensity and photobleaching analysis of the GFP-tagged proteins indicate that the GST-Kif7-CC is an obligate dimer and Gli2-ZF is a monomer (FIGS. 8C & 8D). We performed the bio-layer interferometry (BLI) assay by immobilizing the ligand, Kif7-CC, to the BLI sensor through a GST-tag and using Gli2-ZF as the analyte, and found that these two proteins bind directly with a K_{d} of 48±5 nM (FIG. 1C). Kif7-SCC also binds Gli2-ZF with a similar K_{d} of 69±20 nM, indicating that the first coiled-coil dimerization domain of Kif7 is sufficient for binding to Gli2 (FIG. 1C). We also verified that Kif7-CC-GFP and Gli2-ZF-GFP bind with similar affinity as the untagged proteins (FIG. 8E). Due to higher stability and yield of the Kif7-CC compared to the Kif7-SCC protein, we used the Kif7-CC for further biochemical analysis.

To determine the stoichiometry of the complex (whether one Kif7-CC dimer bind one or two molecules of monomeric Gli2-ZF), we used fluorescence intensity analyses. Size exclusion chromatography performed with a pre-mixed solution of the two GFP tagged proteins shows that the Kif7-CC-GFP:Gli2-ZF-GFP complex elutes as a single peak with a molecular mass greater than either protein on the same column (FIG. 1D). The complex was analyzed by two methods. First, SDS-PAGE followed by in-gel fluorescent intensity analysis of the fraction containing the complex revealed that the stoichiometry of interaction was 1:2, suggesting that one molecule of monomeric Gli2-ZF binds to one Kif7-CC dimer. Second, quantitative western blot with fluorescently labeled anti-GFP antibody and densitometric analysis independently confirmed the stoichiometry of the complex (FIG. 8F).

Together, these biochemical analyses of the Kif7-Gli interaction show that Kif7 uses its coiled-coil dimerization domain, not the canonical C-terminal ‘cargo’ binding domain, to engage Gli2 with high affinity, while Gli2 uses its DNA binding domain to bind Kif7 dimer. Thus, the domain of the Gli transcription factor that mediates its interaction with DNA in the nucleus is repurposed to interact with a kinesin in the cytoplasm.

### The Coiled-Coil Dimerization Domain of Kif7 is a DNA Structural Mimic for Gli Binding

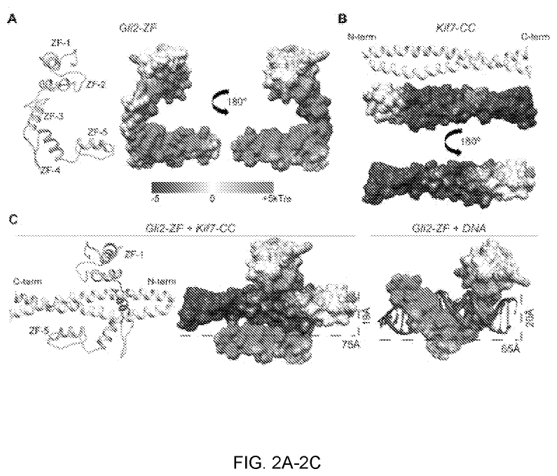

To gain insight into the structural basis of the Kif7-SCC:Gli2-ZF complex and how the DNA binding domain of Gli binds the coiled-coil dimerization domain of Kif7, we performed comparative homology modeling. The structure of Gli2-ZF domain was modelled using the X-ray structure of the zinc-finger domain of Gli1 isoform (PDB: 2GLI) (Pavletich and Pabo, 1993 Science, 261, 1701-1707) as a template (FIG. 2A). This model could be generated with high confidence due to the >95% sequence similarity between Gli1-ZF (235-387aa) and Gli2-ZF (437-589aa). Modeling of the coiled-coil domain of Kif7 yielded a homodimer (FIG. 2B). The large number of X-ray structures of coiled-coil domains make it possible to model this tertiary structure with high accuracy.

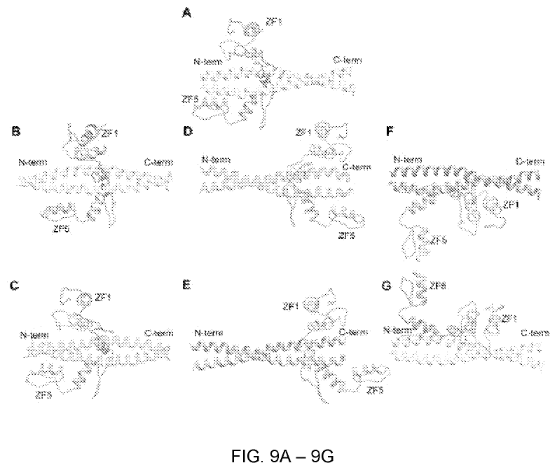

The complex between Gli1 and DNA (PDB: 2GLI) is characterized by the positively charged zinc-finger domain of Gli1 wrapping around the major groove of the rod-like DNA double-helix. Extensive electrostatic contacts exist between the negatively charged phosphate backbone of DNA and the second and third zinc-fingers in Gli (Pavletich and Pabo, 1993, Science, 261, 1701-1707). The structural model of Gli2-ZF is nearly identical to Gli1-ZF (FIG. 2A). Analysis of the structural model of Kif7-SCC revealed two striking features. First, the overall size and shape of Kif7-SCC is similar to DNA. Second, analysis of the electrostatic surface potential (APBS server) of Kif7-SCC shows a highly negatively charged rod-shaped surface, similar to the DNA backbone (FIG. 2B). We hypothesized that the models of the individual interacting domains of Gli2 and Kif7 might allow for interaction with size and surface charge complementarity similar to the Gli-DNA complex. Therefore, we modelled the structure of the complex using the structural models of Kif7-SCC and Gli2-ZF as inputs to the protein-protein docking ClusPro 2.0 server (Kozakov et al., Nature protocols, 12, 255, 2017). The top ranked outputs for the model of the Kif7-Gli2 complex indicate that the positively charged interface of Gli2 zinc-fingers clamp the negatively charged Kif7-SCC rod-like domain (FIG. 2C & FIG. 9) in a manner similar to the Gli-DNA complex. FIG. 9 shows other high confidence structural models of the Kif7-CC-Gli2-ZF protein complex from docking analysis (ClusPro 2.0). Gli2-ZF model is represented in salmon colored ribbon diagram and Kif7-CC models represented in blue and green colored ribbon diagrams in the different models.

The stark similarities in electrostatic surface charge complementarity, molecular dimensions, and mode of binding of Gli2-ZF with DNA and Kif7-SCC (FIG. 2C) indicated that Kif7 may be a DNA molecular mimic for Gli binding in the cytoplasm.

### Experimental Validation of the Structural Model of the Kif7-CC:Gli2-ZF Complex

We used three different approaches to test the structural model of the interaction between Kif7-CC and Gli2-ZF.

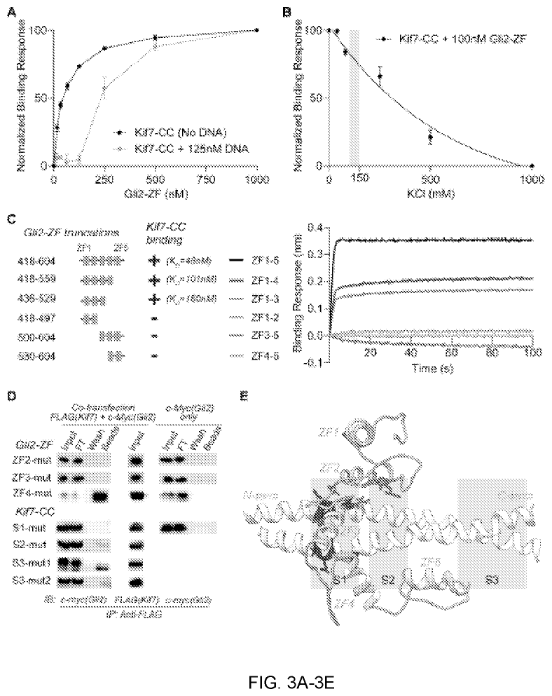

First, we examined whether Kif7-CC and DNA compete for the same binding site on Gli2, which would be expected if Kif7-CC were a DNA mimic. To test this, we measured the binding between Gli2-ZF and Kif7-CC in the presence of a Gli-specific target dsDNA (TRE-2S) (Dan et al., Journal of virology, 73, 3258-3263, 1999). In the presence of 125 nM TRE-2S DNA there was no detectable binding between Kif7-CC and Gli2-ZF until the analyte concentration was above 125 nM Gli2-ZF (FIG. 3A). This suggests that DNA-bound Gli2-ZF cannot bind Kif7-CC and that these interactions are mutually exclusive.

Second, to confirm that complex formation between Kif7-CC and Gli2-ZF is driven by electrostatic interactions, we performed the BLI binding assay with Kif7-CC and 125 nM Gli2-ZF at varying KCl concentrations ranging from 10 mM to 1M (FIG. 3B). We observed that the binding response exhibited a decreasing trend with increase in KCl concentration, while the high-affinity binding interaction was maintained at physiological ionic strength (˜125-150 mM KCl).

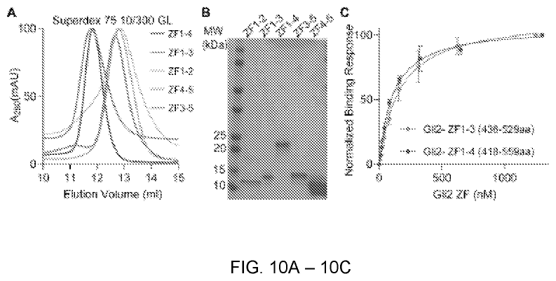

Third, we performed a series of site-directed mutagenesis experiments to further confirm the structural model and map the residues of Gli2-ZF and Kif7-CC required for complex formation. We created five Gli2-ZF truncations containing different groups of zinc-fingers (FIG. 3C; FIGS. 10A & 10B). Only two of these truncations, Gli2-ZF-1-4 (K_{d}=101±16 nM) and Gli2 ZF1-3 (K_{d}=160±40 nM) were able to bind to Kif7-CC, indicating the requirement for zinc-fingers 2 and 3 in tandem for Kif7-CC binding (FIG. 3C & FIG. 10C). This is similar to the Gli-DNA interaction where ZF2 and ZF3 are critical for making contacts with the phosphate backbone of DNA (Pavletich and Pabo, Science, 261, 1701-1707, 1993).

To narrow down the residues that participate in binding, we analyzed the model of the Kif7-SCC:Gli2-ZF complex using PDBsum server (De Beer et al., Nucleic acids research, 42, D292-D296, 2014). We performed alanine scanning mutagenesis of the charged residues on Gli2-ZF that came up as part of the binding interface in the PDBsum analysis and analyzed their binding with pull-down assay (FIG. 3D). Like the truncation studies, mutation of residues in Gli2 ZF-2 (H493A/R496A) and ZF-3 (H503A/R516A/K521A) abolished binding to Kif7-CC whereas those in ZF-4 (R550A/K552A/R556A) did not affect binding. Interestingly, the residues H493 (ZF2), R516 (ZF3) and K512 (ZF3) also engage in phosphate backbone contacts with DNA (Pavletich and Pabo, Science, 261, 1701-1707, 1993), suggesting that the same subset of amino acids interact with Kif7-CC. Analysis of the interaction surface on Kif7-CC revealed three patches of negatively charged amino acid residues (labeled as S1, S2 and S3 in FIG. 3E) that are potential Gli2-ZF binding residues. We systematically mutated the residues to alanine and performed the pull-down assay with Gli2-ZF. Kif7-CC S1 (E500A/E501A/E502A/D505A) mutant abolished binding to Gli2-ZF, while the S2 (E511A/E515A) and S3 (E526A/E529A & E530A/R535A) mutants did not. This suggests that negatively charged residues included in S1 that lie approximately at the center of the coiled-coil rod of Kif7 form the primary contact point for Gli binding (FIG. 3D).

Taken together, the results from DNA competition assay, ionic strength-dependence and the mutagenesis studies validate the model for the Kif7-SCC:Gli2-ZF complex (FIG. 3E), and confirm that the coiled-coil domain of Kif7 acts as a DNA mimic. The center of this DNA-like rod forms the Gli2 interaction site with zinc-fingers 2 and 3 being most critical for making charge-charge contacts, as seen previously in binding of Gli to DNA.

### GIi2-ZF Increases the Microtubule-Binding Affinity of Kif7, Thereby Increasing its Own Recruitment to Microtubules

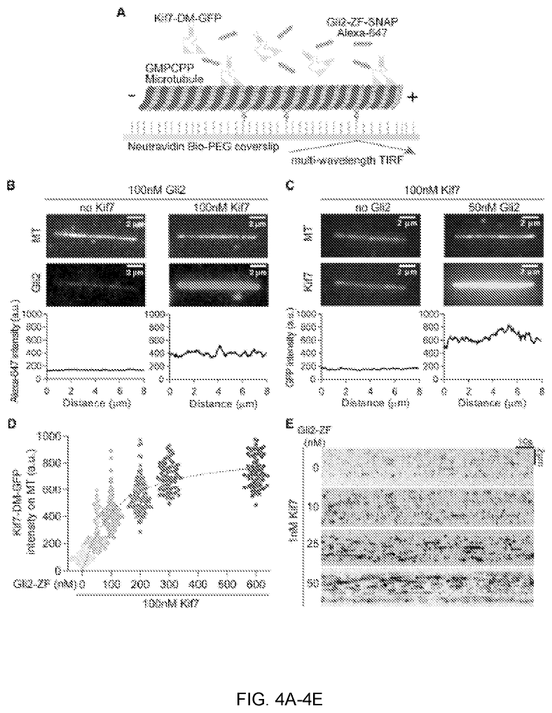

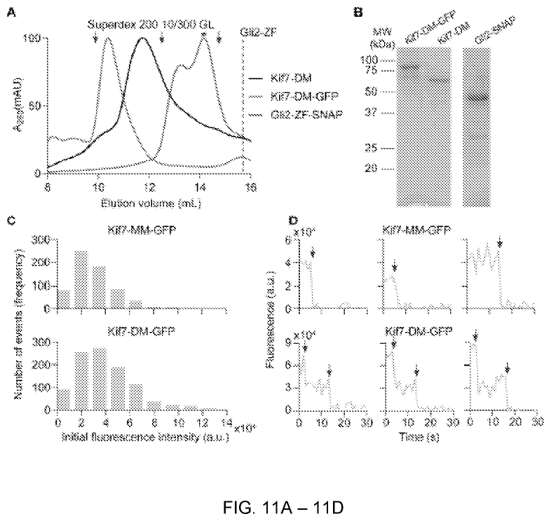

To examine whether the Kif7-Gli interaction described above is sufficient for recruiting Gli to microtubules, we reconstituted Gli and Kif7 on microtubules and examined their activity using an in vitro TIRF microscopy assay. Briefly, fluorescently labeled (Rhodamine/HiLyte-647) and biotinylated microtubule seeds, polymerized with the non-hydrolyzable GTP analog GMPCPP, were immobilized on a glass coverslip through neutravidin-biotin linkages (FIG. 4A). GMPCPP was chosen as it mimics the GTP-form of tubulin present at microtubule ends (Jiang et al., Developmental cell, 49, 711-730. e8 2019). GFP-tagged recombinant dimeric Kif7 protein (1-543aa, referred to as Kif7-DM-GFP; 100 nM) and ATP were added to the chamber either in the absence or presence of Alexa-647 labeled Gli2-ZF-SNAP (100 nM). A Gli2 control was used to measure non-specific binding of Gli2-ZF to microtubules. Single molecule photobleaching of purified Kif7-DM confirmed that it is a dimer (FIGS. 11A-D). Overlay of images of the Alexa-647 and rhodamine channel showed 3-fold increase over background in the intensity of Gli2 on microtubules under these experimental conditions (FIG. 4B). This suggests that Kif7 binding localizes Gli to microtubules.

Interestingly, we observed that the presence of 100 nM Gli2-ZF results in >5-fold increase in the Kif7 fluorescence intensity on microtubules (FIG. 4C). At a constant Kif7-DM-GFP concentration (100 nM), a dose dependent increase in the GFP fluorescence intensity is observed with increasing Gli2-ZF concentrations until it reaches saturation (FIG. 4D). A similar increase in the Kif7-DM-GFP fluorescence intensity was also seen when Gli2-ZF-SNAP-Alexa-647 was used in this assay. Next, to further examine the effect of Gli on the Kif7-microtubule interaction, we measured the residence time of Kif7-DM-GFP on microtubules at single molecule resolution (FIG. 4E). Addition of increasing concentrations of Gli2-ZF resulted in an increase in the lifetimes of single molecules of Kif7-DM-GFP on the microtubule (FIG. 4E). Therefore, our results indicate that Gli2-ZF increases the microtubule-binding affinity of Kif7 by decreasing the off-rates of single Kif7-DM molecules from microtubules.

Thus, the interaction of Kif7 with Gli is not only sufficient to recruit Gli onto microtubules but it also positively regulates the microtubule-binding of Kif7 by increasing the dwell time of the motor on microtubules. This positive regulation in turn increases the amount of Gli2 recruited to microtubules. Hence, the interaction between Gli2 and Kif7 has the synergistic consequence of concentrating both proteins to higher levels on microtubules.

Dual-Site Interaction of Gli2-ZF with the Coiled-Coil and Motor Domain of Kif7 is Necessary for their Synergistic Regulation on Microtubules

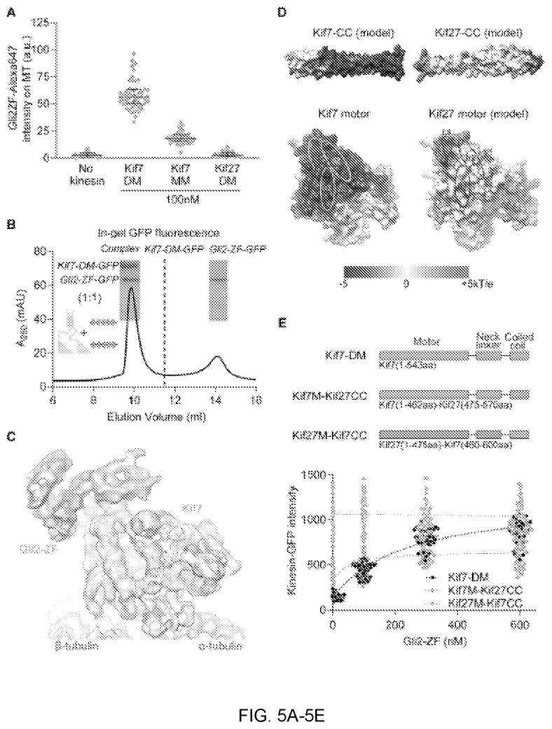

In Kif7, the motor and the coiled-coil domains are separated by an unusually long neck-linker domain (˜120 aa). We were, therefore, interested in examining how Gli-binding to Kif7 at the coiled-coil domain alters the interaction between microtubule and the Kif7 motor domain. One possibility is that Gli2-ZF binding induces specific long-distance conformational changes in the motor domain. Another possibility is that Gli2 may directly engage the Kif7 motor domain via another binding site. To distinguish between these possibilities, we examined the binding of Gli2-ZF-SNAP-Alexa-647 to microtubule-bound monomeric Kif7 motor domain (1-386aa, referred to as Kif7-MM), that lacks the coiled-coil Gli interaction site and compared it to Kif7-DM. Analysis of the Alexa-647 intensity reveals that microtubule-bound Kif7-DM recruits Gli2-ZF at levels that are ˜9 fold higher than Gli2 alone. The microtubule-bound Kif7-MM also binds Gli2-ZF, albeit to a lesser extent when compared to the Kif7-DM (˜3 fold above the background fluorescence of Gli alone on microtubules) (FIG. 5A). Additionally, we failed to detect any binding between the monomeric Kif7-MM and Gli2-ZF at concentrations as high as 12 μM Gli2-ZF in the absence of microtubules in the BLI assay. These data suggest that the motor domain of Kif7 likely forms another Gli-interaction site, but this interaction requires the microtubule or possibly another mechanism, such as dimerization, that brings the motor domains in proximity.

The observation that Gli2-ZF can bind to both the coiled-coil and motor domain of Kif7, suggests two possible modes of interaction: i) one Gli2-ZF molecule binds to both sites on Kif7 simultaneously or ii) multiple Gli2-ZF molecules independently bind the coiled-coil and the motor domains of Kif7. To resolve this, we re-visited the stoichiometry experiment with GFP-tagged versions of Gli2-ZF and Kif7-DM (FIG. 5B). This experiment was performed in the absence of microtubules. The stoichiometry of Kif7-DM-GFP:Gli2-ZF-GFP in the complexed fraction was 1:1, suggesting there are two molecules of Gli2-ZF per Kif7-DM (FIG. 5B). Since one Gli2-ZF is bound to the coiled-coil dimer of Kif7, we deduce that the second molecule binds proximal to the Kif7 motor domains.

The stoichiometry and TIRF experiments show that (a) the coiled-coil domain of Kif7 binds one Gli2-ZF molecule with high affinity, (b) dimeric Kif7-DM binds two molecules of Gli2-ZF, and (c) individual monomeric motor domains of Kif7 do not have a high-affinity for Gli2-ZF except when the motors are bound to microtubules. Together these results suggest that in addition to the high affinity interaction site on Kif7-CC, a second Gli2-ZF interaction site is formed by Kif7 motor domains. Requisite for binding to the second site is dimerization of the kinesin heads or the presence of microtubules, which may serve to orient the motor heads appropriately for interaction.

### Visualization of the Second Gli Binding Site on Kif7 by Cryo-EM

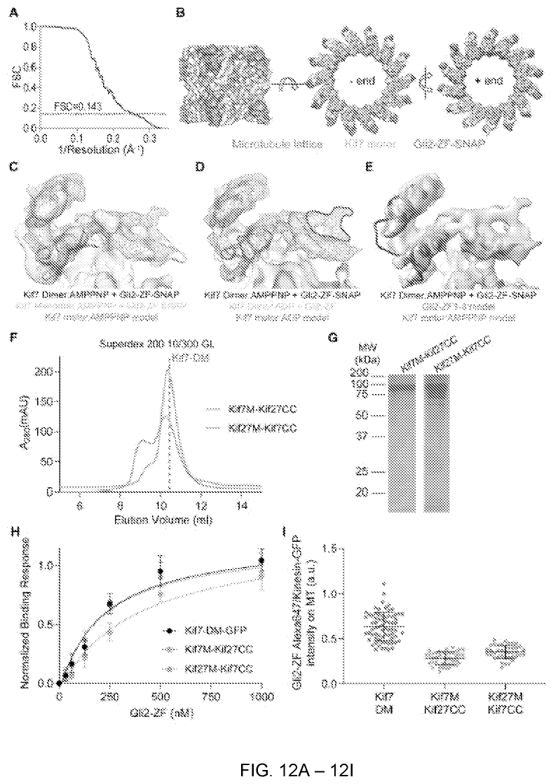

To gain structural insight into the Gli2-ZF:Kif7 motor interaction we obtained cryo-EM reconstructions of Kif7-DM bound to microtubules in the presence of Gli2-ZF-SNAP and the non-hydrolysable ATP analogue, AMP-PNP (3.9 Å resolution, FIG. 5C, FIG. 12A and Table 1). Comparisons of the cryo-EM reconstruction with a map of microtubule-bound Kif7 motor domain in the AMP-PNP state (PDB:6MLR, EMD:9141) showed that the motor domain adopts the same conformation while binding to microtubules in the presence and absence of Gli2-ZF (green in FIG. 5C and FIG. 12B). While no additional density was seen on the microtubule lattice, an extra region of density contacting the motor domain of Kif7 was observed in the presence of Gli2-ZF-SNAP (pink in FIG. 5C and FIG. 12B). Close inspection reveals that this extra density lies close to loop L6, helices α-2 and α-3 on the Kif7 motor domain and away from the nucleotide-binding pocket as well as the microtubule-binding interface of the motor (FIG. 12E). This extra density contacting the motor domain could either arise from the neck-linker and coiled-coil domains of Kif7 or the Gli2-ZF-SNAP protein. Additionally, it is well established that under saturating motor concentrations needed for cryo-EM reconstructions, the density of only a single motor head can be resolved (Hoenger et al., Journal of molecular biology, 297, 1087-1103, 2000, Hirose et al., The EMBO Journal, 19, 5308-5314, 2000). Hence, we do not expect to see the entire Gli2-ZF interaction site formed by both motor heads and the coiled-coil domain in a dimer.

To resolve the origin of the extra density we generated cryo-EM reconstructions of the following microtubule-bound complexes: (i) Kif7-MM in the AMP-PNP state with Gli2-ZF-SNAP at 4.8 Å resolution, and (ii) Kif7-DM in the ADP state with Gli2-ZF, at 4.3 Å resolution (Table 1). The presence of extra density in the Kif7-MM:Gli2-SNAP map at the same position as in Kif7-DM:Gli2-SNAP map ruled out the contribution of Kif7-coiled-coil and neck-linker domains (FIG. 12C). Comparison of the Kif7-DM:Gli2-ZF and Kif7-DM:Gli2-SNAP maps confirmed that a large part of the extra density arose from Gli2-ZF (FIG. 12D). Superposition of the cryo-EM maps obtained in the presence of Gli2-ZF and Gli2-ZF-SNAP was used to unambiguously assign the density corresponding to Gli2-ZF (FIG. 12D). We were able to build a structural model of the Gli2-ZF into this extra density by fitting two complete and one partial zinc-fingers (FIG. 12E). Together these results confirm that in addition to the high affinity interaction site on Kif7-CC, there is a second interaction site for Gli2-ZF proximal to the Kif7 motor domain.

### Kif7 Homolog Kif27 Lacks the Structural Determinants Specifying the Gli2-Kif7 Interaction

Kif27 is a close homolog of Kif7 and these two proteins are thought to arise from a gene duplication event. Unlike Kif7, Kif27 plays a role in motile cilium biogenesis and is not thought to be involved in Hedgehog signaling (He et al., Trends in cell biology, 27, 110-125, 2017). We wondered if the structural determinants of Gli binding are conserved or dissimilar between Kif7 and Kif27. To address this, we first modelled the motor domain (1-398aa) and the coiled-coil dimerization domains (480-550aa) of Kif27 and determined their electrostatic surface potential using APBS server (FIG. 5D). The electrostatic surface potential of Kif27 coiled-coil domain is overall neutral in contrast to that of the Kif7 coiled-coil domain. Similarly, the electrostatic surface potential of the Kif27 motor domain is less negatively charged compared to that of Kif7 motor domain. Of special interest are the regions marked in brown/green (helix α-2, α-3 and loop L6), lying in the Gli2-ZF binding region, that present a less negative surface potential in Kif27 compared to Kif7. Consistent with this, we do not observe recruitment of Gli2-ZF on microtubules by the dimeric Kif27-DM-GFP (1-580aa) protein in our TIRF-microscopy reconstitution assay (FIG. 5A).

Thus, the structural determinants that specify the Kif7-Gli interaction are absent in a close homolog and another ciliary kinesin Kif27. This may indicate a specific adaptation in Kif7 for Gli binding and Hedgehog signaling.

Gli2-ZF Interaction with the Coiled-Coil and Motor Domains of Kif7 is Essential for Regulating the Accumulation of Both Proteins on Microtubules

To address which of the two Gli-interaction sites of Kif7 plays a role in modulating Kif7 microtubule binding, we generated two chimeric proteins where we swapped either the motor and neck-linker (M) or the coiled-coil (CC) domains of Kif7 and Kif27 (Kif7M-Kif27CC and Kif27M-Kif7CC) (FIG. 5E). Since Kif27 does not bind Gli2-ZF (FIG. 5A), the chimeras are dimeric kinesins with single Gli-interaction sites on either the motor domain (Kif7M-Kif27CC) or the coiled-coil domain (Kif27M-Kif7CC). Size-exclusion chromatography show that the two chimeras (Kif7M-Kif27CC & Kif27M-Kif7CC) form stable homogeneous dimers similar to Kif7-DM (FIGS. 12F & 12G). The binding affinity for each of the two Gli interaction sites on Kif7-DM could be determined using BLI (FIG. 12H). We compared the two chimeras to Kif7-DM in TIRF microscopy-based microtubule binding assay. As expected, both chimeras showed decrease (3-fold: Kif7M-Kif27CC; 2-fold: Kif27M-Kif7CC) in Gli recruitment on microtubules compared to Kif7-DM as measured by Alexa-647 intensity per GFP intensity (FIG. 12I). Next, we compared how the microtubule binding of the chimeras were regulated by Gli with respect to Kif7-DM. To our surprise 100 nM Kif7M-Kif27CC alone, in the absence of Gli-ZF, showed ˜10 times higher binding to microtubules compared to 100 nM Kif7-DM as measured by their GFP intensity on microtubules (FIG. 5E; red). Addition of Gli did not result in any further increase in the amount of Kif7M-Kif27CC chimera bound to microtubules. While the Kif27M-Kif7CC chimera also exhibited ˜3 times higher binding to microtubules compared to Kif7-DM without Gli (FIG. 5E; blue), addition of increasing amounts of Gli2-ZF resulted in a slight increase in the microtubule-associated intensity of Kif27M-Kif7CC, which saturates at a lower level compared to Kif7-DM and Kif7M-Kif27CC. The Kif7-DM intensity increased in a dose dependent manner as expected (FIG. 5E; black). Thus, the Gli-dependent enhancement of Kif7 binding to microtubules occurs to a much lower extent in the absence of the Kif7-CC interaction site. These data suggest that both interaction sites on Kif7 are required for the maximal synergy between Gli and Kif7. Any changes that abolish either of these interactions, like in the chimeras, drives the system to high binding but removes the simultaneous and synergistic increase in the accumulation of both proteins on microtubules. Thus, the two Gli-interaction sites on Kif7 form a regulatable module that is responsive to Gli levels in the system.

### Synergistic Regulation of Gli-Kif7 Promotes Kif7 Localization to Cytosolic Microtubules and the Distal Cilium Tip

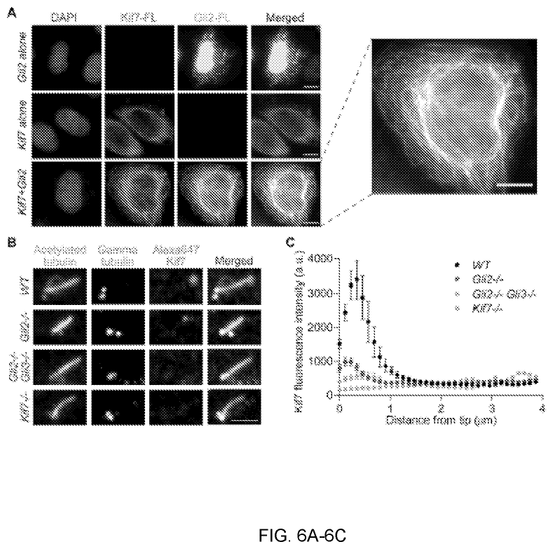

We next investigated the relationship between full-length Gli2 and Kif7 in HeLa cells by analyzing the cellular localization of transiently overexpressed full-length mNeonGreen-Gli2 and full-length Kif7-mRuby. Kif7-FL-mRuby alone showed punctate localization throughout the cytoplasm (FIG. 6A, panel 2). Co-transfection of Kif7-FL-mRuby with mNeonGreen-Gli2-FL revealed binding of both Kif7 and Gli2 along filamentous microtubules in the cytosol (FIG. 6A, panel 3).

Kif7 and Gli2 are known to accumulate at the cilium tip in response to Hedgehog (Hh) pathway activation, and their interaction is essential for proper recruitment of Gli to the cilium tip (Liu et al., Science signaling, 7, ra117-ra117, 2014, He et al., Nature cell biology, 16, 663-672, 2014). However, our in vitro findings suggested that the cilium localization of Kif7 may in turn be Gli-dependent. To examine this further, we measured the amount of Kif7 at the cilium tip in Gli2−/− and Gli2−/−Gli3−/− cell lines (immortalized MEFs) compared to wild-type (WT) MEFs, upon stimulation with the Smoothened agonist SAG (a small molecule activator of Hh pathway). For this analysis, we use a Kif7 antibody directly labeled with Alexa-Fluor-647 for quantitative intensity measurements in immunostaining experiments. We found that the amount of Kif7 at cilia tips during Hh pathway activation depends on Gli (FIGS. 6B & C). Gli2−/− cells showed ˜4-fold less Kif7 in the cilia (integrated intensity under the curve analysis up to 1.5 μm from the cilia tips), whereas Gli2−/− Gli3−/− cells showed 5-fold less Kif7 at the cilia tips compared to WT cells (FIG. 6C). Kif7−/− cells were used as a negative control and showed no Kif7 at the cilium tip.

Together these experimental findings suggest that Gli regulates cellular localization of Kif7 on microtubules and at the cilium tip, and therefore dictates its own as well as Kif7 recruitment to microtubule-structures in cells.

Re-Engineering the DNA-Mimicking Coiled-Coil Domain of Kif7 as a Tool for Sequestration of Gli Away from the Nucleus

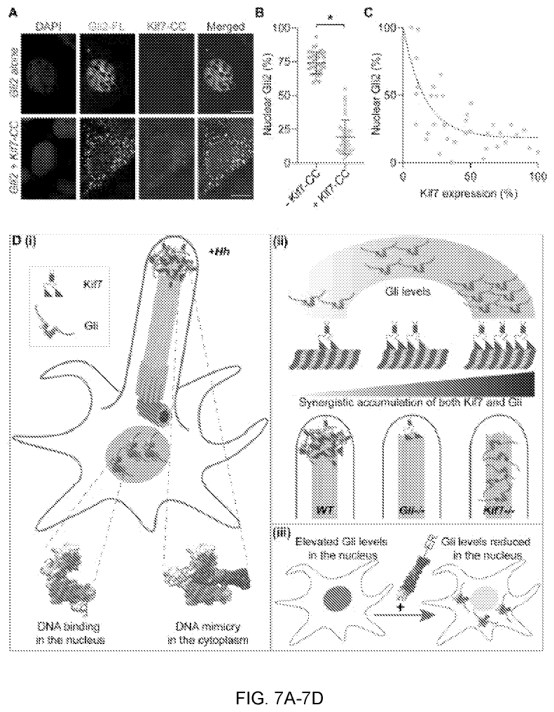

Small coiled-coil domains and helical peptides have the potential to be developed as inhibitors or cellular sequestration tools to tune the levels of a transcription factor in the nucleus. Given the high affinity of the Kif7-CC for Gli2, we wondered if this domain could be retooled to sequester Gli away from the nucleus. We first performed proof-of-principle experiments to test this idea. For these experiments, we used a dual expression vector expressing both Kif7-CC-mRuby and full-length mNeonGreen-Gli2 simultaneously from a single mRNA. The Kif7-CC was engineered to contain an endoplasmic reticulum (ER) localization tag at the C-terminus. The identical vector expressing mNeonGreen-Gli2 alone served as a control. These constructs were transfected in HeLa cells and the amount of Gli2 in the nucleus was measured as a percentage of total Gli expression levels as measured by the mNeonGreen intensity. The localization of mNeonGreen-Gli2 in control experiments was largely nuclear (>75%) (FIG. 7A, panel 1 & B). However, in the presence of ER-localized Kif7-CC-mRuby the nuclear mNeonGreen-Gli2 decreased to <25% (FIG. 7B). Colocalization of fluorescent signals for Gli2 and Kif7-CC showed that ER-linked Kif7-CC-mRuby sequesters Gli2 in the cytoplasm (FIG. 7A, panel 2). We also found that the sequestration of Gli2 in the cytoplasm (and thus away from the nucleus) depended on the level of Kif7-CC-mRuby expression in these cells (FIG. 7C). Cells with higher Kif7-CC expression showed lower levels of nuclear Gli2 and hence higher levels of cytoplasmic Gli2 sequestration. These findings establish that the Kif7-CC peptide can be re-tooled for Gli sequestration in the cytoplasm.

### Re-Engineering for Sequestration of Endogenous and Over-Expressed Gli in the Cytoplasm and Inhibition of Nuclear and Cilium Localization

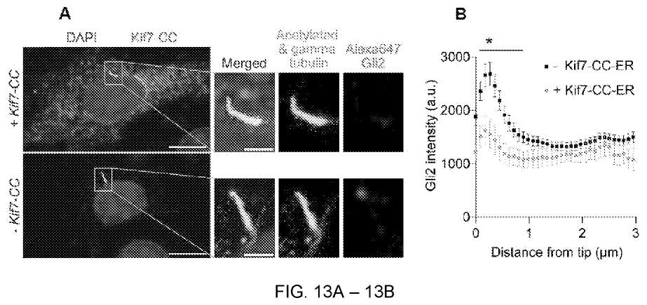

To test if the ER-tagged Kif7-CC could inhibit the cilium tip localization of Gli2 in response to Hedgehog pathway activation we transfected this construct into NIH3T3 cells. The cilium tip localization of Gli2 in Kif7-CC-ER transfected cells, induced with the Hedgehog pathway agonist SAG, was measured using a directly labeled Gli2 antibody that was further cleaned using Gli2−/− MEFs. Our results show that the Kif7-CC-ER inhibits the levels of Gli2 at the cilia tips during pathway activation compared to non-transfected wild type cells (FIG. 13). These findings show that the Kif7-CC peptide can be re-engineered for sequestration of endogenous and over-expressed Gli in the cytoplasm and inhibit its nuclear and cilium localization.

The ciliary kinesin Kif7 has emerged as a regulator of Gli activity in vertebrates that forms the molecular link between Hedgehog signaling pathway and the microtubule cytoskeleton. In this study, we discovered that the coiled-coil dimerization domain of Kif7 is a DNA mimic for interaction with the DNA-binding zinc-finger domain of Gli in the cytoplasm (FIG. 7Di). The Kif7-Gli interaction both recruits Gli to microtubules and increases the lifetimes of Kif7 on microtubules. This feedback regulation results in synergistic accumulation of both proteins on microtubules and requires a second Gli interaction site on the Kif7 motor domain (FIG. 7Dii). Our findings reveal DNA-mimicry by a coiled-coil domain as a cytoskeletal-tethering mechanism for regulating the transcription factor Gli. Proof-of-principle experiments show that this interaction mode can be co-opted for sequestrating Gli away from the nucleus (FIG. 7Diii), thus offering a molecule for specific inhibition of Gli-mediated transcriptional activity when the Hedgehog pathway is erroneously activated.

### Kif7 Coiled-Coil Domain is a Cytoplasmic DNA Mimic for Gli Interaction

Our results show that Gli binding domain in Kif7 is a coiled-coil that has striking size, shape, and electrostatic surface charge similarity with double stranded DNA. Thus, the zinc-finger domain of Gli2 binds DNA in the nucleus and is repurposed for Kif7-mediated cytoplasmic localization through the same structural design principle. Therefore, DNA mimicry by the Kif7 coiled-coil is the first example of a coiled-coil domain in an endogenous eukaryotic protein used to bind the DNA-binding domain of a transcription factor and regulate its function in the cytoplasm. Our observations indicate that cytoskeletal proteins can be integral in regulating other nuclear proteins, especially transcription factors, in the cytoplasm, by mimicking double stranded DNA.

The Coiled-Coil Domain of Kif7 Acts as a Molecular Rheostat that Regulates the Accumulation of Both Kif7 and Gli on Microtubules

Our results show that the coiled-coil domain in Kif7 is more than a Gli-recruitment element. It plays a role in ensuring the synergistic accumulation of both Gli and Kif7 on microtubules in a Gli-concentration dependent manner. We find that such a graded response to Gli levels requires a second interaction site located within the Kif7 motor domain. When either of these Gli interaction sites is abolished, Kif7 is constitutively bound to microtubules more tightly and the Kif7-microtubule interaction is no longer responsive to Gli concentration. At the molecular level, our data indicate that this may arise from inhibition of the Kif7-microtubule interaction by the coiled-coil domain of Kif7. Gli binding to both the coiled-coil and motor interaction sites on Kif7 alleviates this inhibition to elicit the maximum dynamic range of the feedback response. Our results suggest that Gli binding to Kif7 increases its lifetime on microtubules. Moreover, the comparison of Gli-interaction sites on the coiled-coil and motor domains of Kif7 and Kif27 validates that Kif7 and not Kif27 is the mammalian Costal2 ortholog. Together, our results suggest that the coiled-coil domain of Kif7 acts as a molecular rheostat, varying the microtubule binding affinity of the Kif7-Gli complex in a Gli-dependent manner, thus modulating the Hh signal output.

### Implication of Synergistic Kif7-Gli Feedback Mechanism for Hedgehog Signaling

According to our model Kif7 is not a stable but a dynamically regulated Gli-recruitment platform at the cilium tip, providing a mechanism for Kif7 concentrate Gli at the cilium tip upon Hh pathway activation. The interaction between Kif7 and Gli increases the affinity between Kif7 and microtubules, which in turn enhances the concentration of Gli, thus setting up a positive feedback loop. Consistent with this, Gli knockout cell lines have significantly reduced Kif7 at the cilia tips during Hh pathway activation. Thus, by adjusting the levels of Kif7 at the cilium tip, Gli acts as a master regulator of its own recruitment to the cilia tip for its activation. Like other transcription factors, Gli is a low copy number and tightly regulated protein, hence the synergistic feedback between Kif7-Gli and Kif7-microtubule interactions may be advantageous in rapidly concentrating Gli, to the cilium tip upon Hh activation. A similar strategy may be used by Kif7 to concentrate and promote the formation of Gli repressor at the base of the cilium when the Hh pathway is off. Furthermore, given that the nuclear localization signal in Gli overlaps with the Kif7-binding site, Kif7 may potentially occlude the nuclear import of the active form of Gli.

### Re-Engineered Kif7 Coiled-Coil as a Potential Tool for Gli Inhibition

Gli is the final downstream effector molecule of the Hh signaling pathway and it is therefore a target for intervention of the pathway activity. Aberrant activation of Gli has been reported in multiple human cancers including basal cell carcinomas and glioblastomas through both Hh-dependent and independent mechanisms (Matise and Joyner, Oncogene, 18, 7852-7859, 1999, i Altaba et al., Nature Reviews Cancer, 2, 361-372, 2002, Hui and Angers, Annual review of cell and developmental biology, 27, 513-537, 2011). A peptide inhibitor that directly targets Gli has tremendous therapeutic potential in various cancers with Gli overexpression irrespective of their etiology. Our biochemical analyses of Kif7-Gli interaction and proof-of-principle sequestration experiments suggest that a small coiled-coil peptide from Kif7 can be exploited to sequester Gli in the cytoplasm and restrict its entry into the nucleus. The structural analyses of the Kif7-Gli interaction opens the possibility of a new site on Gli that can be exploited for drug design.

Collectively, our study provides critical insights into mechanisms employed to link the Hh signaling pathway and the cytoskeleton and reports the discovery of DNA-mimicry for regulating nuclear proteins in the cytoplasm of eukaryotes.

### Experimental Model and Subject Details

Recombinant proteins were overexpressed in Sf9 cells in accordance with the Bac-to-Bac® Baculovirus virus expression system. Recombinant proteins expressed in E. coli Rosetta (DE3) cells were induced with the addition of 250 μM IPTG after an OD of 0.6. Cells were grown for 18 hours at 4° C. Transient protein expression in HeLa cells and analysis of cilia in MEFs have been detailed in the section headed “Cell Culture and Immunofluorescence”.

### Method Details

### Pull-Down Assay

Plasmids for the pull-down assay were constructed by cloning human Kif7 and Gli2 sequences into pCMV-BICEP™-4 expression vector followed by truncations using InFusion HD Cloning Kit (Takara). Kif7 fragments were cloned at the MCS1 to express FLAG-tagged Kif7 protein and Gli2 fragments were cloned at the MCS2 to express c-myc-tagged Gli2 protein. Point mutations were made using the Q5® Site-Directed Mutagenesis Kit (NEB). Expi293F™ cells were maintained in Expi293 Expression medium and cultured under 7% CO₂, 93% air condition at 37° C. with 220 rpm shaking. Expi293 cells were transfected with plasmids using ExpiFectamine™ 293 Reagent, grown for 18-22 hours followed by addition of ExpiFectamine™ 293 Transfection Enhancer 1 and grown for another 24 hours. Next the cells were harvested by centrifugation at 2500×g for 5 min, washed with phosphate buffered saline (PBS) and resuspended in lysis buffer (25 mM Tris-HCl, 75 mM NaCl, 1 mM MgCl₂, 0.5 mM EDTA, 100 μM ATP, 1 mM DTT, 0.1% TritonX100 and Halt™ protease inhibitor cocktail; pH=7.5). Cells were lysed by a short sonication and the lysate was cleared by centrifugation at 125000×g for 30 mins. Anti-FLAG M2 magnetic beads pre-equilibrated in the lysis buffer was incubated with the cleared cell lysate (labeled ‘input’) for 1 hr at 4° C. DynaMag-Spin was used to collect the supernatant labeled ‘flow through’ and the beads were washed with the buffer 5 times. The last supernatant from the washes was used as the ‘wash’ sample. The final ‘bead’ sample was prepared by resuspending the beads in ˜50ul of buffer. The input, flow through, wash and bead samples were run on SDS-polyacrylamide gel and subjected to Western blotting. Binding was detected using anti-c-myc antibody and the expression of the bait-protein was detected from the input sample with anti-FLAG antibody.

### Protein Expression and Purification

The N-terminal fragment of Kif7 (Uniprot Q2M1P5) (Kif7-DM 1-543aa) was cloned into a pFastBac expression vector (Thermo) that included a tobacco etch virus (TEV) protease cleavable N-terminal 6×His-tag and SUMOstar solubilization tag. To determine which residues to swap in the chimeras, we first performed homology modeling of the two kinesins using PROMALS 3D (UTSW). We complimented this with modeling of Kif7 and Kif27 CC domains using COILS (EMBL) and PairCoil (MIT) to make the most accurate coiled-coil substitution possible. For the Kif7M-Kif27-CC chimera, Kif7 1-462aa was used for the motor and Kif27 475-570aa was used for the coiled-coil. For the Kif27M-Kif7-CC chimera, Kif27 1-475aa was used for the motor and Kif7 460-600aa was used for the coiled-coil. Dimeric Kif7 N-terminal constructs (Kif7-DM 1-543aa) and the Kif7-Kif27 chimeric constructs were expressed in SF9 insect cell line using the Bac-to-Bac® Baculovirus Expressions System (Thermo) with cells grown in HyClone CCM3 SFM (GE Life Sciences) and expressed from P3 virus for 72H at 27° C. Monomeric Kif7 constructs (Kif7-MM 1-398aa) containing the motor domain was cloned into a modified pET-21-a expression vector that contained a TEV protease cleavable N terminal 6×His-tag. Kif7 monomers were expressed in BL21 (DE3) Rosetta (Millipore) E. coli at 18° C. with 0.25 mM IPTG for 18-20 H. Dimeric and monomeric kinesin pellets containing the motor domain of Kif7 were lysed by short sonication in buffer A (50 mM phosphate pH 8.0 300 mM NaCl 5% glycerol, 1 mM MgCl₂ and 25 mM imidazole) supplemented with 0.15% tween, 0.5% Igepal, 100 μM ATP 2 mM TCEP, 1 mM PMSF, 75 U benzonase and 1×HALT (Thermo). Lysate was cleared by ultracentrifugation and supernatant was incubated with Ni-NTA for 1 H. Resin was washed with buffer A supplemented with 20 μM ATP and 0.5 mM TCEP and eluted with 400 mM imidazole with 100 μM ATP. Peak fractions were pooled and, if needed, cleaved overnight by TEV (1/30 w/w) at 4° C. Proteins were further purified by size exclusion chromatography (Superdex 200 10/300GL) in 50 mM HEPES pH 7.4 300 mM NaCl 5% glycerol 5 mM B-Me 1 mM MgCl₂ and 100 μM ATP and frozen in liquid nitrogen. Dimerization of a given construct was determinable by gel filtration profiles.

The coiled-coil dimerization domain fragment of Kif7, (Kif7-CC 460-600aa) was cloned into a modified pGEX vector that contained a human rhinovirus (HRV) protease cleavable N terminal GST-tag. The Kif7 SCC construct (aa488-540) was ordered as a gene-fragment (Genscript) and cloned into pETDuet-1 by InFusion HD Cloning Kit (Takara). Kif7-CC and Kif7-SCC were expressed in BL21 (DE3) Rosetta (Millipore) E. coli at 18° C. with 0.25 mM IPTG for 18-20 hr. The cell pellets were lysed by short sonication in buffer B (PBS pH 7.3, 5% glycerol) supplemented with 0.15% tween, 0.5% Igepal, 100 μM ATP, 2 mM TCEP, 1 mM PMSF, 75 U benzonase and 1×HALT (Thermo). Lysate was cleared by ultracentrifugation and supernatant was incubated with GST-4B beads (GE Life Sciences) for 1 H. Resin was washed with buffer B supplemented with 0.5 mM TCEP and eluted with 10 mM reduced glutathione in 40 mM PIPES pH 7.3 150 mM NaCl. Peak fractions were pooled and, if needed, cleaved overnight by GST-HRV-3C (1/40 w/w) at 4° C. Proteins were further purified by size exclusion chromatography (Superdex 200 10/300GL) in 40 mM PIPES pH 7.3 150 mM NaCl 5% glycerol 2 mM TCEP and frozen in liquid nitrogen.

The zinc-finger domain of Gli2 (Uniprot P10070) (Gli2-ZF 418-604aa) was cloned into a modified pET-Duet-1 expression vector that contained an HRV protease cleavable N terminal 6×His-tag. Truncations were made using the InFusion HD Cloning Kit (Takara). The same construct was cloned into a modified pET-Duet-1 vector that contained an HRV protease cleavable N terminal 6×His-tag, and a C-terminal SNAP/GFP tag. Gli2-ZF protein was expressed in BL21 (DE3) Rosetta (Millipore) E. coli at 18° C. with 0.25 mM IPTG for 18-20 hr. Gli2-ZF pellets were lysed by short sonication in buffer C (40 mM PIPES pH 7.3 150 mM NaCl) supplemented with 0.15% tween, 0.5% Igepal, 2 mM TCEP, 1 mM PMSF, 300 U benzonase and 1× HALT (Thermo). Lysate was cleared by ultracentrifugation and supernatant was incubated with Ni-NTA for 1 H. Resin was washed with buffer A supplemented with 0.5 mM TCEP and eluted with 400 mM imidazole. Peak fractions were pooled and, if needed, cleaved overnight by GST-HRV-3C (1/40 w/w) at 4° C. Proteins were further purified by ion-exchange chromatography using a Heparin column; Gli2-ZF proteins are DNA binding and generally elute from the heparin column around 1M NaCl. Proteins were further purified by size exclusion chromatography (Superdex 75 10/300GL and Superdex 200 10/300GL) in 40 mM PIPES pH 7.3 150 mM NaCl 5% glycerol 2 mM TCEP and frozen in liquid nitrogen.

All proteins were greater than 95% pure and eluted as a single peak from the size exclusion chromatography column.

### Bio-Layer Interferometry Assays

BLI experiments were performed in an Octet Red 96 instrument (ForteBio). To quantify Kif7-Gli2 binding, Experiments were performed in an assay buffer containing 40 mM TRIS pH7.3, 40 mM KCl, 40 μM ATP 1 mM MgCl₂, 0.12% tween and 0.5 mM TCEP. In the simplest assay, dimeric GST-Kif7-CC at a concentration of 120 nM is immobilized on an anti-GST biosensor chip. Unbound Kif7 is washed away with buffer, and the chips are then dipped into buffer with a range of Gli2-ZF concentrations from 0-1 μM to allow for association of the two proteins. After 300 seconds, they are then moved back into a well with just buffer to allow dissociation for 400 seconds. The equilibrium response of each sensor from three independent repeats at each Gli2 concentration were normalized and plotted against Gli2 concentration and fitted with a Hill function to obtain a Kₜ. The DNA competition experiments were performed in the same way with one notable exception: 125 μM TRE-2S DNA (a Gli2 DNA binding target) was held constant along the Gli2 titration from 0-1 μM. The assays were also performed with HIS-tagged Kif7-DM and the Kif7-Kif27 chimeric proteins, requiring the use of an anti-penta-HIS biosensor chip, and referencing sensors to account for non-specific binding of Gli2 to the HIS sensor. A flipped assay, using HIS-tagged Gli2 on the sensor and a range of Kif7 concentrations in buffer, was also conducted.

### Kit7-GIi2 Complex Stoichiometry Determination

The Kif7-CC and Kif7-DM proteins were not amenable to dynamic light scattering analysis and so in order to determine the stoichiometry of the Kif7-Gli2 complexes, we purified GFP-tagged constructs of Kif7-DM, Kif7-CC, and Gli2-ZF. Next, the Kif7 (Kif7-CC or Kif7-DM) and Gli2 were mixed at high concentration with Gli2-ZF in molar excess (125 μM Kif7 and 750 μM Gli2-ZF) and incubated for 15 minutes on ice. After incubation, 200 μL of this mix was injected into a Superdex 200 10/300 GL column. Fractions that contained complexed protein were determined by elution volume shift in the gel filtration chromatogram. Complex containing fractions were boiled and run in SDS-PAGE and quantified in two ways: (1) Scan of an unstained SDS-PAGE gel at 488 nm, and (2) Scan of a Western blot using a directly labeled 488 nM anti-GFP antibody (Santa Cruz). Both methods were quantified using an Amersham™ Typhoon Scanner and ImageJ for intensity analysis.

### Structural Modeling

Primary sequences of the zinc-finger domain of human Gli2 (UniprotKB P10070) (Gli2-ZF, 437-589aa), the coiled-coil domain of human Kif7 (UniprotKB Q2M1P5) (Kif7-SCC, 481-543aa), the coiled-coil domain of human Kif27 (UniprotKB Q86VH2) (Kif27-CC, 475-570aa) and the N-terminal motor domain of human Kif27 (UniprotKB Q86VH2) (Kif27M, 1-475aa) were retrieved from the database in FASTA format. A preliminary search for homologs to serve as templates for modeling was performed with BLAST (Camacho et al., BMC bioinformatics, 10, 421, 2009) and HHBlits (Remmert et al., Nature methods, 9,173-175, 2012) against the SWISS-MODEL template library (SMTL). The following templates with the highest quality predictions (based on target-template alignment) were selected for homology modeling: (i) Gli1 zinc finger (PDB: 2GLI) for Gli2-ZF (ii) ROCK1 coiled-coil (PDB: 300Z) for Kif7-CC and Kif27-CC (iv) Kif7 motor domain (PDB: 6MLQ) for Kif27M. The 3D structure of these protein domains was built using ProMod3 in the SWISS-MODEL server (Guex et al., Electrophoresis, 30, S162-S173, 2009, Waterhouse et al., Nucleic acids research, 46, W296-W303, 2018). The global and per-residue model quality was assessed using GMQE and QMEAN scoring function (Benkert et al., Bioinformatics, 27, 343-350, 2011). The structural models were visualized in UCSF Chimera (Pettersen et al., J Comput Chem, 25, 1605-12, 2004). Electrostatic potential of the surfaces were generated from the APBS server (Jurrus et al., Protein Sci, 27, 112-128, 2018).

For obtaining the structural model of the Kif7-SCC:Gli2-ZF protein complex, the Gli2-ZF model was docked on the Kif7-CC model using an automated server, ClusPro 2.0 (Kozakov et al., Nature protocols, 12, 255, 2017), in which receptor was Kif7-SCC and Gli2-ZF was used as a ligand. Based on different desolvation and electrostatic potential, ClusPro 2.0 can differentiate thousands of conformations of the protein. The generated conformations were further categorized through clustering and 8 most fit structures (which are found to be closest to native structure from X-ray crystallography results) were considered. The interface areas in the structural model of the protein complex were calculated by the PDBsum webserver (De Beer et al., Nucleic acids research, 42, D292-D296, 2014). These contact residues were mutated to Ala for binding analysis to assess the predicted models.

### Total Internal Reflection Fluorescence Microscopy Assays

In vitro TIRF-based microscopy experiments were carried out as described in Subramanian et al., 2013 (Subramanian et al., Cell, 154, 377-390, 2013). Microscope chambers were constructed using a 24×60 mm PEG-Biotin coated glass slide and 18×18 mm PEG coated glass slide separated by double-sided tape to create two channels for exchange of solutions. Standard assay buffer was 1× BRB80 (80 mM KCl-PIPES at pH 6.8, 2 mMMgCl2 and 1 mMEGTA), 1 mMATP, 1 mMGTP, 0.1% methylcellulose and 3% sucrose. Images were acquired using NIS-Elements (Nikon) and analyzed using ImageJ.

Microtubule binding assay: X-rhodamine/HiLyte 647 (1:10 labeled to unlabeled) and biotin (1:10 labeled to unlabeled) labeled microtubules were polymerized in the presence of GMPCPP, a non-hydrolysable GTP-analogue, and immobilized on a neutravidin coated glass coverslip. Coverslips were briefly incubated with casein to block non-specific surface binding before addition of 100 nM kinesin and/or varying concentrations of GIi2-ZF in assay buffer and antifade reagent (25 mMglucose, 40 mg/ml glucose oxidase, 35 mg/ml catalase and 0.5% p-mercaptoethanol). Images were acquired from multiple fields of the same chamber.

Single molecule imaging: For single molecule imaging, microtubule binding assays were performed with altered conditions: 1 nM Kif7DM-GFP with varying concentrations of GIi2-ZF. Images were collected at 300 ms intervals on an ANDOR iXon Ultra EMCCD camera.

### Cryo-EM Sample Preparation

For grid preparation, all Kif7-GIi2 preparations were diluted using BRB80 (80 mM 1,4-piperazinediethanesulfonic acid [PIPES], pH 6.8, 1 mM MgCl₂, 1 mM ethylene glycol tetraacetic acid [EGTA]). Porcine brain microtubules were prepared from frozen aliquots of 5 mg/ml tubulin (Cytoskeleton, Denver, CO) in polymerization buffer (BRB80; 80 mM PIPES, pH 6.8, 1 mM EGTA, 2 mM MgCl₂, 3 mM GMP-CPP, 10% dimethyl sulfoxide (DMSO)) at 37° C. for 2-3 hours. The polymerized microtubules were then incubated at room temperature for several hours or overnight before use. The following day or several hours later the microtubules were spun down on a bench top centrifuge 14.00 RPM 10 min to pellet. The pellet was resuspended in polymerization buffer without DMSO. The Kif7 dimer and monomer preparations were made by mixing the Kif7 with the GIi2 or GIi2-SNAP for 1 hour prior to incubation with the microtubules. Kif7-dimer (0.36 mg/ml) plus GIi2 (0.2 mg/ml) in BRB plus 2 mM ADP, 2 mM MgcI2, Kif7-monomer (0.45 mg/mL) or Kif7 dimer plus GIi2_SNAP (0.30 mg/mL) plus BRB80 with 1 mM AMPPNP and 1 mM MgCl₂. All microtubule samples were prepared on 1.2/1.3 400-mesh grids (Electron Microscopy Services) Grids were glow-discharged before sample application. The cryo-samples were prepared using a manual plunger, which was placed in a homemade humidity chamber that varied between 80 and 90% relative humidity. A 4-μl amount of the microtubules at ˜0.5 μM in BRB80 was allowed to absorb for 1 min, and then 4 μl of the different Kif7-dimer or monomer plus GIi2 with and without SNAP were incubated with microtubules on the grid. After a short incubation of 2 min, the sample was blotted (from the back side of the grid) and plunged into liquid ethane.

### EM Image Acquisition and Data Processing

Images of frozen-hydrated Kif7-microtubule complexes (see Supplemental Table1) were collected on a Titan Krios (FEI, Hillsboro, OR) operating at 300 keV or an Arctica (FEI, Hillsboro, OR) equipped with a K2 Summit direct electron detector (Gatan, Pleasanton, CA). The data were acquired using the Leginon automated data acquisition (Suloway et al., J Struct Biol, 151, 41-60, 2005, Wilson-Kubalek et al., Mol Biol Cell, 27, 1197-203, 2016). Image processing was performed within the Appion processing environment (Lander et al., J Struct Biol, 166, 95-102, 2009, Wilson-Kubalek et al., Mol Biol Cell, 27, 1197-203, 2016). Movies were collected at a nominal magnification of 29000× and 36000× with a physical pixel size of 1.03 and 1.15 Å/pixel respectively. Movies were acquired using a dose rate of ˜4.2 and 5.6 electrons/pixel/second over 9 and 8 seconds yielding a cumulative dose of ˜36 and ˜34 electrons/Å² (respectively). The MotionCor frame alignment program (Li et al., Nature methods, 10, 584-590, 2013, Hirschi et al., Nature, 550, 411-414, 2017) was used to motion-correct. Aligned images were used for CTF determination using CTFFIND4 (Rohou and Grigorieff, J Struct Biol, 192, 216-21, 2015) and only micrographs yielding CC estimates better than 0.5 at 4 Å resolution were kept. Microtubule segments were manually selected, and overlapping segments were extracted with a spacing of 80 Å along the filament. Binned boxed segments (2.05 Å/pixel, 240 pixel box size for the Krios data and 2.30 Å/pixel, 192 pixel box size for the Arctica data) were then subjected to reference-free 2D classification using multivariate statistical analysis (MSA) and multi-reference alignment (MRA) (Ogura et al., J Struct Biol, 143, 185-200, 2003, Hirschi et al., Nature, 550, 411-414, 2017). Particles in classes that did not clearly show an 80 Å layer line were excluded from further processing.

### Cryo-EM 3D Reconstruction

Undecorated 13,14- and 15-protofilament microtubule densities (Sui and Downing, Structure, 18, 1022-31, 2010) were used as initial models for all preliminary reconstructions. We used the IHRSR procedure (Egelman, J Struct Biol, 157, 83-94, 2007) for multimodel projection matching of microtubule specimens with various numbers of protofilaments (Alushin et al., Cell, 157, 1117-29, 2014), using libraries from the EMAN2 image processing package (Tang et al., J Struct Biol, 157, 38-46, 2007). After each round of projection matching, an asymmetric back projection is generated of aligned segments, and the helical parameters (rise and twist) describing the monomeric tubulin lattice are calculated. These helical parameters are used to generate and average 13, 14 and 15 symmetry-related copies of the asymmetric reconstruction, and the resulting models were used for projection matching during the next round of refinement. The number of particles falling into the different helical families varied. Helical families that had enough segments were further refined. Final refinement of microtubule segment alignment parameters was performed in FREALIGN (Grigorieff, J Struct Biol, 157, 117-25, 2007) without further refinement of helical parameters. The data is unbinned during refinement. The boxsize of the Krios data was binned from 480 to 380 reducing the pixel size from 1.03 to 1.3 Å/pixel. FSC curves were used to estimate the resolution of each reconstruction, using a cutoff of 0.143. To better display the high-resolution features of the 3D map shown, we applied a B-factor of 150 Å, using the program bfactor (http://grigoriefflab.janelia.org).

### Model Building in the EM Map

The EM-derived structure of AMPPNP-Kif7 bound to microtubules (PDB: 6 MLR, EMD: 9141) was fit as a rigid body into the density for AMPPNP-Kif7 dimer bound to microtubules in the presence of Gli2-SNAP, using the ‘Fit in Map’ utility in UCSF Chimera (Pettersen et al., J Comput Chem, 25, 1605-12, 2004). To locate the density corresponding to the Gli2 protein, cryo-EM maps for ADP-Kif7 dimer with Gli2, AMPPNP-Kif7 monomer with Gli2-SNAP and AMPPNP-Kif7 dimer with Gli2-SNAP were superposed via the Kif7 motor domains, and the minimal density common to all three maps was identified. To construct a structural model for Gli2 to fit the density, individual zinc fingers were first extracted from the crystal structure of a five-finger Gli1 in complex with DNA (PDB:2GLI). Next, various combinations of zinc fingers were fit into the minimal density corresponding to Gli2 and the best model was identified through its model-map correlation coefficient and visual inspection. Since the density corresponding to each zinc finger could be fit by any one of the five zinc-fingers of Gli2, the partial structure for Gli2 was modeled as a Poly-Ala sequence. Finally, flexible fitting for the structure comprising of Gli2 zinc fingers, AMPPNP-bound Kif7 motor and α-β tubulin heterodimer was performed with phenix.real_space_refine (Afonine et al., Computational Crystallography Newsletter, 4, 43-44, 2013). The final structure has a model-map correlation of 0.84, bond length RMSD of 0.007 Å and bond angle RMSD of 0.91°. Only 1 residue (0.08%) is an outlier in the Ramachandran map. Cryo-EM reconstructions and their associated models were superposed using the UCSF Chimera “Fit in map” tool (Pettersen et al., J Comput Chem, 25, 1605-12, 2004).

### Kif7 Antibody Generation and Labeling

The coiled-coil dimerization domain fragment of Kif7, (Kif7-CC 460-600aa) expressed and purified for the biochemistry experiments was used to produce antibodies in rabbits from Covance Inc. (Princeton, NJ). The Kif7 antiserum was affinity-purified using antigen conjugated CNBr-Sepharose resins. The Kif7 antibody was further ‘cleaned’ by incubation with fixed Kif7−/− MEFs to reduce non-specific staining in immunofluorescence experiments and labeled with Alex Fluor 647 dye using Molecular Probes kit.

### Cell Culture, Transfections, and Immunofluorescence

HeLa cells were obtained from Robert Kingston (Massachusetts General Hospital) and MEFs (WT, Gli2−/−, Gli2−/−Gli3−/−, Kif7−/−) were obtained from Kathryn Anderson (Sloan Kettering Institute), Robert Lipinski (University of Wisconsin), Adrian Salic (Harvard Medical School), and Stephane Angers (University of Toronto). Both HeLa cells and MEFs were maintained in high-glucose Dulbecco's modified Eagle's medium (DMEM) supplemented with 10% fetal bovine serum (FBS), sodium pyruvate (1 mM) and L-glutamine (2 mM). Cells were cultured on clean coverslips under the following conditions: 5% CO₂, 95% air condition at 37° C.

HeLa cells were transfected with plasmids using jetPrime transfection reagent and incubated for 18-22 hours. Next the cells were fixed using a mixture of methanol and acetone (1:1 in volume) for 10 minutes in −20° C., washed with washing buffer (PBS+0.05% Tween 20) and mounted on 25 mm×75 mm glass slides with ProLong Diamond Antifade Mountant with DAPI (Thermo Fisher). Z-stacks were acquired on an inverted Nikon confocal microscope using laser illumination source (405 nm, 488 nm and 561 nm channels corresponding to DAPI, Gli2-Neongreen and Kif7 respectively) with a pinhole of 0.5.

For experiments with MEFs, cells were grown to confluency and then serum-starved with 0.2% FBS in DMEM to induce ciliogenesis for 24 hours, followed by treatment with 500 nM SAG for 12-18 hours before being fixed for immunofluorescence. Cells were fixed using a mixture of methanol and acetone (1:1 in volume) for 10 minutes in −20° C., washed with washing buffer (PBS+0.05% Tween 20) for 3 times, blocked with blocking buffer (PBS+2% BSA; OmniPur BSA; EMD Millipore) for one hour at room temperature. Samples were probed overnight at 4° C. with the following primary antibodies (diluted in blocking buffer): Alexa Fluor 647 nm labeled Kif7 antibody (1:10) (in-house), Cyanine3 labeled polyclonal γ-tubulin antibody (1:1000) (Thermo Fisher) and Alexa Fluo 488 nm labeled acetylated α-tubulin antibody (1:500) (Santa Cruz Biotechnology). Samples were mounted on a 25 mm×75 mm glass with ProLong Diamond Antifade Mountant (Thermo Fisher). Z-stacks were acquired on an inverted Nikon confocal microscope using laser illumination sourc.3e (488 nm, 561 nm and 647 nm channels corresponding to acetylated α-tubulin, γ-tubulin and Kif7 respectively) with a pinhole of 1. Z-projections were generated by Sum of images from planes that included the entire cilium.

### Quantification and Statistical Analysis

ImageJ was used to assess GFP/Alexa 647 fluorescence intensities on microtubules. For all average intensity per pixel values recorded, a rectangular area along the microtubule was selected with a width of 3 pixels. Background intensities were also subtracted locally from regions of interest using the same principle around the selected microtubule. Intensities were not analyzed for microtubules found at the edges of the camera's field of view. Data were analyzed using GraphPad Prism. “N” numbers in all experiments refer to the unique number of microtubules used for the dataset and standard deviations correspond to deviations from the mean. Statistical details can be found in the results section and corresponding figure legends.

ImageJ was used to analyze intensity of protein bands for stoichiometry determination assays in FIGS. 1D, 8F, 5B and 12G. A rectangular box was drawn around the protein band of interest for intensity readout. Local background was subtracted using a 3-pixel radius around the region of interest.

Octet Data Analysis software was used to extract the binding response for BLI data shown in FIGS. 1C, 3A, 3B, 3C, 8E, 10C and 12H. The responses were plotted as a function of protein concentration and fit to Hill curves using GraphPad Prism. The binding responses were normalized between 0 and 100 prior to curve fitting.

For structure fitting, atomic models were fit into the cryo-EM density using UCSF Chimera and model to map correlation coefficients were calculated using the ‘Fit in Map’ utility.

For quantification of Kif7 intensity in the cilia, images of cilia were analyzed on ImageJ. The Alexa647 intensity profile along the length of each cilia was measured from tip to base. The acetylated α-tubulin channel was used as a marker for cilia length and the γ-tubulin channel was used as a marker for the base of the cilia. Intensity profiles of different cilia lengths were aligned by their tips and averaged. The integrated intensity profiles were plotted using GraphPad prism. To compare the Kif7 intensities in each cell type, area under the curve analysis was performed on the integrated intensity profiles from the cilia tip up to 1.51.5 μm.

For cytoplasmic sequestration quantification, images of cells were analyzed on ImageJ. The nucleus boundary points (including the nucleolus pattern) were identified by using automatic thresholding on DAPI channel (Otsu method in Image J). The threshold value was obtained from auto-thresholding the brightest frame of confocal z-stacks images. This threshold value was used to create binary masks on individual frame of Z-stacks. These masks created by applying threshold on the DAPI channel (called “DAPI Nucleus mask”) were used to measure Gli2-neongreen intensity in the nucleus. Finally, the nuclear Gli2-neongreen intensity was integrated after background subtraction by rolling ball (r=100 pixels) in image J. Next, to create a mask in the Gli2-neongreen for the whole cell boundary an ImageJ macro was applied that automatically determined the threshold value with the best-match mask in the nucleus area (“Gli2 Nucleus mask”) with “DAPI Nucleus mask”. The best match was selected by finding the maximum value in XNOR operation between “Gli2 Nucleus mask” and “DAPI Nucleus mask”. With the given threshold value, a binary mask over the whole cell area was created (“Gli2 whole cell mask”) and again the Gli2-neongreen intensity in the whole cell was integrated after background subtraction by rolling ball (r=100 pixels).

TABLE 1
Data collection and reconstruction related FIGS. 5C & 12A-E

| | Ki7-MM-AMPPNP- | Ki7-DM-AMPPNP- | Kif7-DM- |
|---|---|---|---|
| | Gli-SNAP | Gli-SNAP | ADP-Gli |
| | (14 protofilaments) | (14 protofilaments) | (15protofilaments) |
| Data collection | | | |
| Microscope | Titan Krios (FEI) | Titan Krios (FEI) | Arctica (FEI) |
| Voltage (kV) | 300 | 300 | 200 |
| Nominal magnification* | 29,000X | 29,500X | 36,000X |
| Cumulative exposure dose | 36 | 36 | 34 |
| (e⁻ Å⁻²) | | | |
| Exposure rate (e⁻/ | 4.2 | 4.2 | 5.6 |
| pixel/sec) | | | |
| Detector | K2 Summit | K2 Summit | K2 Summit |
| Pixel size (Å)* | 1.03 | 1.03 | 1.15 |
| Defocus range (μm) | 0.04-4.5 | 0.17-4.8 | 0.04-4.5 |
| Average defocus (μm) | 1.18 | 1.29 | 1.17 |
| Micrographs Used | 687 | 1,364 | 2,199 |
| Total extracted helical | 24,677 | 50,639 | 25,474 |
| segment (no.) | | | |
| Refined helical segment | 11,956 | 32,251 | 16,880 |
| (no.) | | | |
| Reconstruction | | | |
| Final helical segments | 9,952 | 25,730 | 16,880 |
| (no.) | | | |
| Symmetry imposed | HP | HP | HP |
| Resolution (global) FSC | 4.8 | 3.89 | 4.3 |
| 0.143 | | | |

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations described herein following, in general, the principles described herein and including such departures from the invention that come within known or customary practice within the art to which the invention pertains and may be applied to the essential features hereinbefore set forth, and follows in the scope of the claims.

Other embodiments are within the claims.

1. A peptide comprising a coiled-coil dimerization domain which exploits DNA mimicry to engage Gli, wherein the peptide is 170 amino acids or fewer.

2. The peptide of claim 1, wherein the coiled-coil dimerization domain comprises an amino acid sequence substantially identical to the coiled-coil dimerization domain of Kif7.

3. The peptide of claim 1, wherein the peptide comprises an amino acid sequence substantially identical to KEDEG AQQLL TLQNQ VARLE EENR DFLAA LEDAM EQYKL QSDRL REQQE EMVEL RLRLE LVRP (SEQ ID NO:3).

4. The peptide of claim 1, wherein the peptide comprises an amino acid sequence substantially identical to DSGIE SASVE DQAAQ GAGGR KEDEG AQQLL TLQNQ VARLE EENRD FLAAL EDAME QYKLQ SDRLR EQQEE MVELR LRLEL VRPGW GGPRL LNGLP PGSFV PRPHT APLGG AHAHV LGMVP PACLP GDEVG SEQRG EQVTN (SEQ ID NO:2).

5. The peptide according to any one of claims 1-4 having a mutation that increases Kif7-Gli binding affinity such as an S1-mutation (E500A, E501A, E502A, D505A), an S2-mutation (E511A, E515A), an S3-mutation 1 (E526A, E529A) or an S3-mutation 2 (E530A, R535A).

6. A peptide according to any one of claims 1-5 further comprising an endoplasmic reticulum tag, a nuclear export signal tag, or a mitochondrial localization tag.

7. Nucleic acid encoding a peptide of claims 1-6.

8. A vector comprising the nucleic acid of claim 7, said vector being capable of directing expression of the protein encoded by the nucleic acid in a vector-containing cell.

9. A cell which contains the nucleic acid of claim 7 or the vector of claim 8.

10. A composition comprising a peptide according to claims 1-6 formulated in a physiologically-acceptable carrier.

11. A composition comprising a nucleic acid according to claim 9 or the vector of claim 10 formulated in a physiologically-acceptable carrier.

12. Use of a polypeptide according to any of claims 1-5 in the manufacture of a medicament for the treatment of cancer in a mammal.

13. A method of treating a cancer in a subject comprising administering a peptide according to any one of the aforementioned claims to the subject in an amount sufficient to inhibit the Gli oncogene in the patient.

14. The method according to claim 13 in which the cancer is a cancer that results from over activation of Gli or the Hedgehog pathway.

15. The method according to claim 13 in which the cancer is selected from glioblastoma, basal cell carcinoma, medulloblastoma, colorectal cancer, prostate cancer, lung cancer and breast cancer.

16. A method of inhibiting Gli-mediated transcriptional activity in a subject having a cancer in which Gli is aberrantly activated comprising administering to the subject a peptide according to any one of the aforementioned claims.

17. A peptide having the amino acid sequence comprising
(SEQ ID NO: 2)
DSGIE SASVE DQAAQ GAGGR KEDEG AQQLL
TLQNQ VARLE EENRD FLAAL EDAME QYKLQ
SDRLR EQQEE MVELR LRLEL VRPGW GGPRL
LNGLP PGSFV PRPHT APLGG AHAHV LGMVP
PACLP GDEVG SEQRG EQVTN.

18. A peptide having the amino acid sequence comprising
(SEQ ID NO: 3)
KEDEG AQQLL TLQNQ VARLE EENR DFLAA
LEDAM EQYKL QSDRL REQQE EMVEL RLRLE
LVRP.

19. A peptide according to claim 17 or 18 having an S1-mutation (E500A, E501A, E502A, D505A), an S2-mutation (E511A, E515A), an S3-mutation 1 (E526A, E529A) or an S3-mutation 2 (E530A, R535A).

20. A peptide according to any one of claims 17-19 further comprising an endoplasmic reticulum tag, a nuclear export signal tag, or a mitochondrial localization tag.

21. Use of a peptide according to any one of claims 17-20 to inhibit the Gli (glioma associated oncogene).

22. A method of treating a cancer in a patient comprising administering a peptide according to any one of claims 17-20 to the patient in an amount sufficient to inhibit the Gli oncogene in the patient.

23. The method according to claim 22 in which the cancer is a cancer that results from over activation of Gli or the Hedgehog pathway.

24. The method according to any one of claims 22-23 in which the cancer is selected from glioblastoma, basal cell carcinoma, medulloblastoma, colorectal cancer, prostate cancer, lung cancer and breast cancer.

25. A method of inhibiting Gli-mediated transcriptional activity in a subject having a cancer in which Gli is aberrantly activated comprising administering to the subject a peptide according to any one of claims 17-20.

26. Use of a peptide according to any one of claims 17-20 to modify intracellular localization and downstream transcriptional activity of Gli.

27. Use of a peptide according to any one of claims 17-20 to modulate the Hedgehog signaling pathway.

28. Use of a peptide according to any one of claims 17-20 conjugate other zinc-finger domain containing transcription factors to the cytoplasmic cytoskeleton to regulating their activity.

29. A Gli sequestration agent for decreasing nuclear Gli comprising a peptide according to any one of claims 17-20.


SEQUENCE LISTING (3 sequences)

SEQ ID NO: 1 - protein, 1343 aa, Homo sapiens
MGLEAQRLPGAEEAPVRVALRVRPLLPKELLHGHQSCLQVEPGLGRVTLGRDRHFGFHVVLAEDAGQEAVYQACVQPLLEAFFEGFNATVFAYGQTGSGKTYTMGEASVASLLEDEQGIVPRAMAEAFKLIDENDLLDCLVHVSYLEVYKEEFRDLLEVGTASRDIQLREDERGNVVLCGVKEVDVEGLDEVLSLLEMGNAARHTGATHLNHLSSRSHTVFTVTLEQRGRAPSRLPRPAPGQLLVSKFHFVDLAGSERVLKTGSTGERLKESIQINSSLLALGNVISALGDPQRRGSHIPYRDSKITRILKDSLGGNAKTVMIACVSPSSSDFDETLNTLNYASRAQNIRNRATVNWRPEAERPPEETASGARGPPRHRSETRIIHRGRRAPGPATASAAAAMRLGAECARYRACTDAAYSLLRELQAEPGLPGAAARKVRDWLCAVEGERSALSSASGPDSGIESASVEDQAAQGAGGRKEDEGAQQLLTLQNQVARLEEENRDFLAALEDAMEQYKLQSDRLREQQEEMVELRLRLELVRPGWGGPRLLNGLPPGSFVPRPHTAPLGGAHAHVLGMVPPACLPGDEVGSEQRGEQVTNGREAGAELLTEVNRLGSGSSAASEEEEEEEEPPRRTLHLRRNRISNCSQRAGARPGSLPERKGPELCLEELDAAIPGSRAVGGSKARVQARQVPPATASEWRLAQAQQKIRELAINIRMKEELIGELVRTGKAAQALNRQHSQRIRELEQEAEQVRAELSEGQRQLRELEGKELQDAGERSRLQEFRRRVAAAQSQVQVLKEKKQATERLVSLSAQSEKRLQELERNVQLMRQQQGQLQRRLREETEQKRRLEAEMSKRQHRVKELELKHEQQQKILKIKTEEIAAFQRKRRSGSNGSVVSLEQQQKIEEQKKWLDQEMEKVLQQRRALEELGEELHKREAILAKKEALMQEKTGLESKRLRSSQALNEDIVRVSSRLEHLEKELSEKSGQLRQGSAQSQQQIRGEIDSLRQEKDSLLKQRLEIDGKLRQGSLLSPEEERTLFQLDEAIEALDAAIEYKNEAITCRQRVLRASASLLSQCEMNLMAKLSYLSSSETRALLCKYFDKVVTLREEQHQQQIAFSELEMQLEEQQRLVYWLEVALERQRLEMDRQLTLQQKEHEQNMQLLLQQSRDHLGEGLADSRRQYEARIQALEKELGRYMWINQELKQKLGGVNAVGHSRGGEKRSLCSEGRQAPGNEDELHLAPELLWLSPLTEGAPRTREETRDLVHAPLPLTWKRSSLCGEEQGSPEELRQREAAEPLVGRVLPVGEAGLPWNFGPLSKPRRELRRASPGMIDVRKNPL

SEQ ID NO: 2 - protein, 140 aa, Homo sapiens
DSGIESASVEDQAAQGAGGRKEDEGAQQLLTLQNQVARLEEENRDFLAALEDAMEQYKLQSDRLREQQEEMVELRLRLELVRPGWGGPRLLNGLPPGSFVPRPHTAPLGGAHAHVLGMVPPACLPGDEVGSEQRGEQVTN

SEQ ID NO: 3 - protein, 63 aa, Homo sapiens
KEDEGAQQLLTLQNQVARLEEENRDFLAALEDAMEQYKLQSDRLREQQEEMVELRLRLELVRP